SEQUENCE LISTING (7 sequences)

SEQ ID NO: 1 - protein, 393 aa, Human herpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 2 - protein, 907 aa, Human herpesvirus 5
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 3 - protein, 904 aa, Human herpesvirus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 4 - protein, 824 aa, Human herpesvirus 2
MEPRPGTSSRADPGPERPPRQTPGTPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 5 - protein, 1318 aa, Human herpesvirus 2
MSAEQRKKKKTTTTTQGRGAEVAMADEDGGRLRAAAETTGGPGSPDPADGPPPTPNPDRRPAARPGFGWHGGPEENEDEADDAAADADADEAAPASGEAVDEPAADGVVSPRQLALLASMVDEAVRTIPSPPPERDGAQEEAARSPSPPRTPSMRADYGEENDDDDDDDDDDDRDAGRWVRGPETTSAVRGAYPDPMASLSPRPPAPRRHHHHHHHRRRRAPRRRSAASDSSKSGSSSSASSASSSASSSSSASASSSDDDDDDDAARAPASAADHAAGGTLGADDEEAGVPARAPGAAPRPSPPRAEPAPARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPAAAAAPLPSAAASPADERAVPAGYGAAGVLAALGRLSAAPASAPAGADDDDDDDGAGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

SEQ ID NO: 6 - protein, 491 aa, Human herpesvirus 5
MESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 7 - protein, 580 aa, Human herpesvirus 5
MESSAKRKMDPDNPDEGPSSKVPRPETPVTKATTFLQTMLRKEVNSQLSLGDPLFPELAEESLKTFEQVTEDCNENPEKDVLTELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRHTNRSLEYKNLPFMIPSMHQVLEEAIKVCKTMQVNNKGIQIIYTRNHEVKNEVDQVRCRLGSMCNLALSTPFLMEHTMPVTHPPDVAQRTADACNDGVKAVWNLKELHTHQLCPRSSDYRNMIIHAATPVDLLGALNLCLPLMQKFPKQVMVRIFSTNQGGFMLPIYETAAKAYAVGQFEKPTETPPEDLDTLSLAIEAAIQDLRNKSQ

(19) United States
(12) Patent Application Publication
(10) Pub. No.: US 2016/0067330 A1
(43) Pub. Date: Mar. 10, 2016

(54) HERPES VIRUS VACCINES AND TREATMENTS

(71) Applicant: The Regents of the University of California, Oakland, CA (US)

(72) Inventors: Deborah H. Spector, La Jolla, CA (US); Christopher S. Morello, Carlsbad, CA (US); Kimberly A. Kraynyak, Encinitas, CA (US)

(21) Appl. No.: 14/850,722

(22) Filed: Sep. 10, 2015

Related U.S. Application Data
(63) Continuation of application No. PCT/US2014/023246, filed on Mar. 11, 2014.
(60) Provisional application No. 61/776,538, filed on Mar. 11, 2013.

(51) Int. Cl. A61K 39/245; C12N 7/00

(52) U.S. Cl. CPC A61K 39/245; C12N 7/00; A61K 2039/55572

(57) ABSTRACT

Provided herein are vaccine compositions and methods of their use in treating or preventing Herpes diseases.

### CROSS-REFERENCES TO RELATED APPLICATIONS

This application claims the benefit of U.S. Provisional Application No. 61/776,538, Filed Mar. 11, 2013, is incorporated herein by reference in its entirety and for all purposes.

### REFERENCE TO A “SEQUENCE LISTING,” A TABLE, OR A COMPUTER PROGRAM LISTING APPENDIX SUBMITTED ON A COMPACT DISK

The Sequence Listing written in file 88654-901546.TXT, created Mar. 9, 2014, 47,085 bytes, machine format IBM-PC, MS-Windows operating system, is hereby incorporated by reference.

### BACKGROUND OF THE INVENTION

Herpes viruses are a leading cause of human disease. Herpes viruses cause lifelong, latent infections that can remain silent for long periods of time, and periodically reactivate. There are 8 human herpesviruses—Herpes Simplex Virus 1 (HSV-1), Herpes Simplex Virus 2 (HSV-2), Varicella-Zoster, Cytomegalovirus, Epstein Barr, Human Herpesvirus 6, Human Herpesvirus 7, and Kaposi's Sarcoma Associated Virus.

The high prevalence of HSV infection in the U.S., together with the medical and psychological impacts of the disease, makes HSV an important candidate for the development of an effective vaccine. Genital herpes (GH) disease is caused by the herpes simplex viruses (HSVs). HSV-2 is the primary cause of recurrent genital lesions and infects between 10 and 50% of the population worldwide, and in the US, it is estimated that 15% of the population is infected (for review, see (29). HSV-1 infects approximately 60% of the US population and is associated with recurrent orolabial lesions, encephalitis, and keratitis, and is an increasingly common cause of primary GH disease (28). Of great importance worldwide are the findings that genital HSV-2 infection significantly increases the risk of acquisition and transmission of HIV-1 (for review, see (7, 8, 11)). Thus, there is a need in the art for treatments of Herpes viruses, including HSV causing GH. Provided herein are solutions to these and other problems in the art.

### BRIEF SUMMARY OF THE INVENTION

Disclosed herein, inter alia, are vaccine compositions and methods of their use in treating a Herpes disease. The vaccine compositions include an inactivated Herpes virus, a recombinant-Herpes virus protein and a vaccine adjuvant. The vaccine composition may be supplied in a kit that includes an administration device.

Also disclosed herein, inter alia, are methods of administering the vaccine compositions described herein, including embodiments thereof, for treating or preventing a Herpes disease. In one aspect, the method includes administering a therapeutically effective amount of a vaccine composition as described herein, including embodiments thereof to treat a Herpes disease. In another aspect, the method includes administering a prophylactically effective amount of a vaccine composition as described herein, including embodiments thereof, to prevent a Herpes disease. The vaccine compositions described herein may provide protection from and treatment of Herpes diseases.

Provided herein are kits that include the vaccine compositions described herein and an administration device.

### BRIEF DESCRIPTION OF THE DRAWINGS

FIG. 1: Addition of HSV-2 glycoprotein D to the vaccine greatly increases protective efficacy and generates sterilizing immunity in guinea pigs (FI-HSV2-formalin inactivated HSV2, AlH=Alhydrogel, MPL=monophosphorylated lipid A, gD2=glycoprotein D2).

### DETAILED DESCRIPTION OF THE INVENTION

The abbreviations used herein have their conventional meaning within the chemical and biological arts.

The term “vaccine” is used according to its plain ordinary meaning within medicine and immunology and refers to a composition including an antigenic component for administration to a subject (e.g. human), which elicits an immune response to the antigenic component. In embodiments a vaccine is a therapeutic. In embodiments, a vaccine is prophylactic. In embodiments a vaccine includes one or more adjuvants.

The term “prime-boost” or “prime boost” as applied to a methodology of administering vaccines is used according to its plain ordinary meaning in virology and immunology and refers to a method of vaccine administration in which a first dose of a vaccine or vaccine component is administered to a subject or patient to begin the administration (prime) and at a later time (e.g. hours, days, weeks, months later) a second vaccine is administered to the same patient or subject (boost). The first and second vaccines may be the same or different but are intended to both elicit an immune response useful in treating or preventing the same disease or condition (e.g. infection by HV, HSV, HHV-1, HHV-2, HHV-3, HHV-4, HHV-5, HHV-6, HHV-7, or HHV-8). In embodiments the prime is a DNA vaccine including one or more viral genes or portions thereof and the boost is a vaccine including one or more viral genes or portions thereof, one or more viral proteins or portions thereof, or one or more inactivated or attenuated viruses. In embodiments, the prime is one or more attenuated or inactivated viruses and the boost is a DNA vaccine including one or more viral genes or portions thereof, one or more viral proteins or portions thereof, or one or more inactivated or attenuated viruses. In embodiments the prime is one or more viral proteins or portions thereof and the boost is a DNA vaccine including one or more viral genes or portions thereof, one or more viral proteins or portions thereof, or one or more inactivated or attenuated viruses. In embodiments, the prime is a vaccine including an inactivated virus (e.g. HV, HSV, HHV-1, HHV-2, HHV-3, HHV-4, HHV-5, HHV-6, HHV-7, or HHV-8) and the boost is a second dose of the same vaccine as the prime.

The term “herpesvirus” or “herpes virus” or “HV” refers to human herpesviruses and may be used, depending on the context, to refer to one, more, or all of the human herpesviruses, including Human Herpesvirus-1 (HHV-1, Herpes Simplex Virus-1, HSV1, HSV-1), HHV-2 (Herpes Simplex Virus-2, HSV2, HSV-2), HHV-3 (Varicella Zoster Virus, VZV), HHV-4 (Epstein-Barr Virus, EBV), HHV-5 (Cytomegalovirus, CMV, HCMV), HHV-6, HHV-7, HHV-8 (Kaposi's Sarcoma-associated Herpesvirus, KSHV). A herpesvirus may be HHV-1. A herpesvirus may be HHV-2. A herpesvirus may be HHV-3. A herpesvirus may be HHV-4. A herpesvirus may be HHV-5. A herpesvirus may be HHV-6. A herpesvirus may be HHV-7. A herpesvirus may be HHV-8. In embodiments, each of the terms HHV-1, HHV-2, HHV-3, HHV-4, HHV-5, HHV-6, HHV-7, and HHV-8 refers to all strains of each respective HHV. In embodiments, each of the terms HHV-1, HHV-2, HHV-3, HHV-4, HHV-5, HHV-6, HHV-7, and HHV-8 refers to a single strain of that HHV. In embodiments, each of the terms HHV-1, HHV-2, HHV-3, HHV-4, HHV-5, HHV-6, HHV-7, and HHV-8 includes mutants of that particular HHV. A herpesvirus may be HSV (HHV-1 and/or HHV-2). A herpesvirus may be human cytomegalovirus (e.g. HCMV). A “Herpes disease” as used herein refers to infection with an HHV as described above.

An “inactivated Herpes virus” is a herpesvirus which is no longer able to produce evidence of growth or infect a subject. An inactivated Herpes virus thus cannot replicate but remains sufficiently intact to elicit an immune response as described herein. A herpes virus may be inactivated using techniques known in the art including but not limited to heat, radiation (e.g. UV, infrared, alpha, beta, gamma, X-ray, visible, microwave), ultrasonic vibration, or chemical treatment (e.g. formalin). The term “chemical treatment” or “chemical inactivation” as applied to inactivation of a virus (e.g. HV, HSV, HHV-1, HHV-2, HHV-3, HHV-4, HHV-5, HHV-6, HHV-7, or HHV-8), refers to a method of inactivating (as defined herein) a virus including contacting the virus with a chemical agent, wherein the contacting causes the inactivation of the virus.

The term “formalin” is used according to its plain ordinary meaning in Chemistry and refers to an aqueous solution including formaldehyde (e.g. 37% by mass). The components of formalin are typically designed to be in sufficient quantities to inactivate a virus. In some embodiments, formalin may include methanol. In some embodiments, formalin does not include methanol.

The terms “Herpes Simplex Virus” and “HSV” are used according to their common meaning and refer to the double stranded DNA viruses Herpes Simplex Virus 1 (HSV1, HSV-1, HHV-1) and Herpes Simplex Virus 2 (HSV2, HSV-2, HHV-2). In embodiments, HSV includes all strains of HSV1 and HSV2. In embodiments, HSV may refer to a single strain of HSV1 or HSV2. In embodiments, HSV includes multiple strains of HSV1 and/or HSV2. In embodiments, HSV includes mutants of HSV1 and/or HSV2. The terms “HSV1” and “HSV2” are used according to their plain ordinary meaning. In embodiments, HSV1 includes all strains or HSV1. In embodiments, HSV1 includes mutants of HSV1. In embodiments, HSV2 includes all strains or HSV2. In embodiments, HSV2 includes mutants of HSV2.

The terms “cytomegalovirus” “HCMV,” and “CMV” are used according to their common meaning and refer to the double stranded DNA virus HHV-5. In embodiments, CMV includes all strains of CMV. In embodiments, CMV refers to a single strain of CMV. In embodiments, CMV includes multiple strains of CMV. In embodiments, CMV includes mutants of CMV.

The term “adjuvant” is used in accordance with its plain ordinary meaning within immunology and refers to a substance that is commonly used as a component of a vaccine. Adjuvants may increase an antigen specific immune response in a subject when administered to the subject with one or more specific antigens as part of a vaccine. An adjuvant may accelerate an immune response to an antigen. An adjuvant may prolong an immune response to an antigen. An adjuvant may enhance an immune response to an antigen. Exemplary adjuvants include, but are not limited to, an aluminum-based mineral salt adjuvant, squalene, a lipopolysaccharide-adjuvant, bacterial cell wall components, molecular cages, a nucleic acid, an oil, a virosome, QS21, or MF59.

The terms “lipopolysaccharide-adjuvant” and “LPS-adjuvant” are used interchangeably herein and refer to a lipopolysaccharide or molecule derived from a lipopolysaccharide commonly employed as part of a vaccine formulation. The LPS-adjuvant may be designed to increase the immune response of a subject when administered to the subject as a component of a vaccine or method of vaccination relative to the absence of the lipopolysaccharide molecule. An LPS-adjuvant may include a portion of an LPS or, in embodiments, an LPS. An LPS-adjuvant may include a modified portion of an LPS (e.g. chemical modification including, for example, additional substituents or bound to a component that is not a portion of an LPS). Thus, in certain instances, an LPS-adjuvant includes a component that is a portion of an LPS and another component that is not a portion of an LPS. An LPS-adjuvant may include a component that corresponds to a portion of an LPS, but which is chemically synthesized. The term “lipopolysaccharide” and “LPS” are used according to their plain meaning in biology, biochemistry, and immunology and refer to a molecule comprising one or more lipids and one or more polysaccharides covalently bonded together. In embodiments, an LPS is a component of the outer membrane of Gram-negative bacteria. In embodiments, an LPS-adjuvant is a monophosphoryl lipid A (MPL) adjuvant (e.g. a monophosphorylated lipid A).

The term “lipid A adjuvant” refers to an adjuvant including the lipid A portion of lipopolysaccharide isolated from the remainder of a lipopolysaccharide molecule. A lipid A adjuvant is an example of an LPS-adjuvant. In embodiments, lipid A includes two glucosamine molecules covalently bonded to fatty acid molecules and including one phosphate molecule covalently bonded to each glucosamine molecule. In embodiments, lipid A includes six fatty acid molecules. In embodiments, lipid A includes six fatty acid molecules each comprising 10 to 16 carbon atoms.

The term “aluminum-based mineral salt adjuvant” refers to an adjuvant including an aluminum salt. An aluminum-based mineral salt adjuvant may include aluminum hydroxide. An aluminum-based mineral salt adjuvant may be aluminum hydroxide. An aluminum-based mineral salt adjuvant may include aluminum phosphate. An aluminum-based mineral salt adjuvant may be aluminum phosphate. An aluminum-based mineral salt adjuvant may include potassium aluminum sulfate. An aluminum-based mineral salt adjuvant may be potassium aluminum sulfate. An aluminum-based mineral salt adjuvant may be an aluminum hydroxide adjuvant. An aluminum-based mineral salt adjuvant may be an aluminum phosphate adjuvant. An aluminum-based mineral salt adjuvant may be a potassium aluminum sulfate adjuvant. An aluminum-based mineral salt adjuvant may be Alum. In embodiments, an aluminum-based mineral salt adjuvant is compound having the formula of CAS no. 21645-51-2. An aluminum-based mineral salt adjuvant may be aluminum hydroxide gel. An aluminum-based mineral salt adjuvant may be aluminum hydroxide gel in the form of a white gelatinous precipitate. In embodiments, an aluminum-based mineral salt adjuvant is CAS no. 7784-30-7. An aluminum-based mineral salt adjuvant may be aluminum phosphate gel. An aluminum-based mineral salt adjuvant may be aluminum phosphate gel in the form of a white gelatinous precipitate.

In embodiments, an aluminum-based mineral salt adjuvant is not Imject Alum Adjuvant™ In embodiments, an aluminum-based mineral salt adjuvant is aluminum hydroxide without magnesium hydroxide. In embodiments, an aluminum-based mineral salt adjuvant is Alhydrogel™ In embodiments, an aluminum-based mineral salt adjuvant is Adju-phos™. In embodiments, an aluminum-based mineral salt adjuvant is not Adju-phos™. An aluminum-based mineral salt adjuvant may be amorphous aluminum hydroxide and not crystalline aluminum hydroxide. An aluminum-based mineral salt adjuvant may include amorphous aluminum and not crystalline aluminum. An aluminum-based mineral salt adjuvant may be crystalline aluminum hydroxide and not amorphous aluminum hydroxide. An aluminum-based mineral salt adjuvant may include crystalline aluminum and not amorphous aluminum. An aluminum-based mineral salt adjuvant may include crystalline aluminum oxyhydroxide. An aluminum-based mineral salt adjuvant may be crystalline aluminum oxyhydroxide. An aluminum-based mineral salt adjuvant may include amorphous aluminum hydroxyphosphate. An aluminum-based mineral salt adjuvant may be amorphous aluminum hydroxyphosphate. An aluminum-based mineral salt adjuvant may include aluminum oxyhydroxide and not aluminum hydroxycarbonate. An aluminum-based mineral salt adjuvant may be aluminum oxyhydroxide and not aluminum hydroxycarbonate. An aluminum-based mineral salt adjuvant may include aluminum oxyhydroxide and not magnesium hydroxide. An aluminum-based mineral salt adjuvant may be aluminum oxyhydroxide and not magnesium hydroxide. In embodiments, an aluminum-based mineral salt adjuvant does not include amorphous aluminum hydroxide in which some hydroxyls are replaced by sulfate anions. An aluminum-based mineral salt adjuvant may include aluminum oxyhydroxide in a Boehmite-like pattern. An aluminum-based mineral salt adjuvant may be aluminum oxyhydroxide in a Boehmite-like pattern. In embodiments of an aluminum-based mineral salt adjuvants described above, the description is of the aluminum-based mineral salt adjuvant prior to inclusion in a vaccine. In embodiments, an aluminum-based mineral salt adjuvant is an aluminum containing adjuvant approved by the FDA for administration to humans. In embodiments, an aluminum-based mineral salt adjuvant is an aluminum hydroxide adjuvant approved for administration to humans by the FDA. In embodiments, an aluminum-based mineral salt adjuvant is an aluminum phosphate adjuvant approved for administration to humans by the FDA.

The term “aluminum hydroxide adjuvant” as used herein refers to the aluminum hydroxide adjuvant that includes aluminum hydroxide. Aluminum hydroxide adjuvants may include those currently used in human vaccines. Aluminum hydroxide adjuvant also may refer to an aluminum hydroxide adjuvant that is currently used in human vaccines and is used in accordance with the use of that term in Hem S. L., Vaccine 23(2007) 4985-4986. In embodiments, an aluminum hydroxide adjuvant includes CAS no. 21645-51-2. An aluminum hydroxide adjuvant may be aluminum hydroxide gel. An aluminum hydroxide adjuvant may be aluminum hydroxide gel in the form of a white gelatinous precipitate. An aluminum hydroxide adjuvant may include aluminum hydroxide and does not include magnesium hydroxide. An aluminum hydroxide adjuvant may be Alhydrogel™. An aluminum hydroxide adjuvant may include crystalline aluminum hydroxide and not amorphous aluminum hydroxide. An aluminum hydroxide adjuvant may include crystalline aluminum and not amorphous aluminum. An aluminum hydroxide adjuvant may include crystalline aluminum oxyhydroxide. An aluminum hydroxide adjuvant may be crystalline aluminum oxyhydroxide. An aluminum hydroxide adjuvant may include aluminum oxyhydroxide and not aluminum hydroxycarbonate. An aluminum hydroxide adjuvant may be aluminum oxyhydroxide and not aluminum hydroxycarbonate. In embodiments, an aluminum hydroxide adjuvant does not include amorphous aluminum hydroxide in which some hydroxyls are replaced by sulfate anions. An aluminum hydroxide adjuvant may include aluminum oxyhydroxide in a Boehmite-like pattern. In embodiments described above, the description is of an aluminum hydroxide adjuvant prior to inclusion in a vaccine.

The term “aluminum phosphate adjuvant” as used herein refers to the aluminum phosphate adjuvant that includes aluminum phosphate. Aluminum phosphate adjuvants may include those currently used in human vaccines. Aluminum phosphate adjuvant may also refer to an aluminum phosphate adjuvant that is currently used in human vaccines and is used in accordance with the use of that term in Hem S. L., Vaccine 23(2007) 4985-4986. In embodiments, an aluminum phosphate adjuvant includes CAS no. 7784-30-7. An aluminum phosphate adjuvant may be aluminum phosphate gel. An aluminum phosphate adjuvant may be aluminum phosphate gel in the form of a white gelatinous precipitate. An aluminum phosphate adjuvant may be Adju-phos™. An aluminum phosphate adjuvant may include amorphous aluminum hydroxyphosphate. In embodiments described above, the description is of an aluminum phosphate adjuvant prior to inclusion in a vaccine.

The term “potassium aluminum sulfate adjuvant” refers to an adjuvant that includes potassium aluminum sulfate.

An “antigen” is used in accordance with its plain ordinary meaning within immunology and refers to a molecule that elicits an immunological response or binds to receptor or antibody thereby eliciting an immunological response. The terms “antigenic” or “antigenicity” are used in accordance with their plain ordinary meaning within immunology and refer an immunological response elicited from an antigen. Thus, “antigenic” and “antigenicity” may refer to specific immunological response from a specified subject. In embodiments, the antigenicity is associated with an immunological response from a bird, reptile, or fish. In embodiments, antigenicity is associated with immunological response from a mammal such as, for example, a mouse, rat, guinea pig, rabbit, cow, dog, cat, chimpanzee or human. In embodiments, antigenicity is associated with immunological response from a mouse, rat, guinea pig, or rabbit. In embodiments, antigenicity is associated with immunological response from a human. The term “antigenic site” therefore refers to a residue of-, a modification of-, a portion of-, or the full-length of-a polynucleotide or polypeptide that elicits an immunological response as described above. In embodiments, an antigenic site is a polypeptide sequence or amino acid residues within a recombinant-Herpes virus protein described herein, including embodiments thereof.

The terms “bind”, “bound”, “binding”, and other verb forms thereof are used in accordance with their plain ordinary meaning within enzymology and biochemistry and refer to the formation of one or more interactions or contacts between two compositions that may optionally interact. Binding may be intermolecular or intramolecular.

The term “deactivated” when used in reference to a recombinant-Herpes virus protein refers to a protein with decreased activity related to the wild type. The decreased activity may be caused by a modification (e.g. chemical modification or amino acid mutation including, for example, substitution or deletion of an amino acid residue), which decreases the activity of the recombinant-Herpes virus protein compared to a corresponding unmodified recombinant-Herpes virus protein. In embodiments, the activity of the recombinant-Herpes virus protein is decreased by at least 20% 30%, 40%, 50%, 60%, 70%, 80%, 90%, 95%, or 100% activity compared to its corresponding unmodified recombinant-Herpes virus protein.

The term “modified-” when used in reference to a recombinant-Herpes virus protein refers to a chemical modification or amino acid mutation of the recombinant-Herpes virus protein including, for example, substitution or deletion of an amino acid residue. The modification may be a “deactivating mutation” (e.g. a mutations which deactivates the protein as described above). The modification may be a modification, for example, which increases expression of the recombinant-Herpes virus protein, increases the solubility of the recombinant-Herpes virus protein, alters the translocation or secretion of a recombinant-Herpes virus protein, alters folding of the recombinant-Herpes virus protein, or otherwise alters the activity and/or function of the recombinant-Herpes virus protein. Modification of proteins described herein may be through chemical modification (e.g. chemical conjugation to residues in the protein, and posttranslational modifications known in the art such as thioesterification, glycosylation, formylation, acetylation, methylation, carboxylation, amidation, phosphorylation, lipodation (i.e. addition of a lipid moiety to a polypeptide), oxidation, or reduction.

As defined herein, the term “inhibition”, “inhibit”, “inhibiting” and the like in reference to an interaction which negatively affects (e.g. decreases) the activity or function of the protein. In embodiments inhibition refers to reduction of a disease or symptoms of disease. In embodiments inhibition refers to reduction of the growth, proliferation, or spread of a virus (e.g. HV (e.g. an HV selected from the group consisting of HHV-1, HHV-2, HHV-3, HHV-4, HHV-5, HHV-6, HHV-7, and HHV-8)). In embodiments inhibition refers to preventing the infection of a subject by a virus (e.g. HV (e.g. an HV selected from the group consisting of HHV-1, HHV-2, HHV-3, HHV-4, HHV-5, HHV-6, HHV-7, and HHV-8)). In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating the signaling pathway or enzymatic activity or the amount of a protein.

The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

A nucleic acid (such as a polynucleotide), amino acid (such as a polypeptide), or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence or polypeptide sequence that does not appear in nature, for example a variant of a naturally occurring gene, is recombinant.

An “endogenous-Herpes virus protein” refers to a protein that is encoded by a Herpes virus and is within or associated with the virus particle. An endogenous-Herpes virus protein may refer to a protein encoded by a Herpes virus, is within or associated with the virus particle, and has function in, for example, forming the envelope, capsid, or matrix (i.e. tegument) of a virus particle, or, for example, in regulating the expression of early or late viral genes (e.g. immediate early proteins, early proteins, early-late proteins, or late proteins). A “corresponding endogenous-Herpes virus protein” refers to an endogenous-Herpes virus protein having sequence identity (e.g. at least 70%, 75%, 80%, 85%, 90%, 95%, 96%, 97%, 98%, 99% or 100%) to a recombinant-Herpes virus protein. In embodiments, the sequence identity refers to the full-length of the polypeptide. In embodiments, the sequence identity refers to a contiguous length of the polypeptide (e.g. about 100, 200, 300, 400, 500, 600, 650, 700, 750, 800, 850, 900, 950, 1000, 1100, 1200, 1300, 1400, 1500, or 1600 amino acids).

A “recombinant-Herpes virus protein” or “rHVP” refers to a recombinantly expressed protein derived from a Herpes virus. A recombinant-Herpes virus protein may have sequence identity to a corresponding endogenous-Herpes virus protein. In embodiments, a recombinant-Herpes virus protein is a Herpes virus protein having function in forming the envelope of a virus particle (e.g. an “envelope protein”). In embodiments, a recombinant-Herpes virus protein is a Herpes virus protein having function in forming the capsid of a virus particle (e.g. a “capsid protein”). In embodiments, a recombinant-Herpes virus protein is a Herpes virus protein having function in forming the matrix or tegument of a virus particle (e.g. a “matrix protein”). In embodiments, a recombinant-Herpes virus protein is a Herpes virus protein having function in regulating expression of immediate early genes (i.e. Herpes virus genes that are the first expressed following infection which may initiate the transition from quiescence) of a Herpes virus (e.g. an “immediate early protein”). In embodiments, a recombinant-Herpes virus protein is a Herpes virus protein having function in regulating expression of early genes of a Herpes virus (e.g. an “early protein”). In embodiments, a recombinant-Herpes virus protein is a Herpes virus protein having function in regulating expression of early-late genes of a Herpes virus (e.g. an “early-late protein”). In embodiments, a recombinant-Herpes virus protein is a Herpes virus protein having function in regulating expression of late genes of a Herpes virus (e.g. an “late protein”). In embodiments, a recombinant-Herpes virus protein is a Herpes virus protein having structural function in a Herpes virus (e.g. a “structural protein”). In embodiments, a recombinant-Herpes virus protein is a Herpes virus protein having non-structural function in a Herpes virus (e.g. a “non-structural protein”). In embodiments, a recombinant-Herpes virus protein is a Herpes virus protein having function in replication of a Herpes virus (e.g. a “replication protein”). Replication proteins include “replication complex proteins,” “proteins essential for virus replication,” and “proteins dispensable for virus replication.”

An envelope protein may be a glycoprotein. Exemplary glycoproteins include, but are not limited to, glycoprotein B (gB) (e.g. YP_{—}081514 (SEQ ID NO: 2): NP_{—}044497.1 (SEQ ID NO: 3)), glycoprotein D (gD) (e.g. NP_{—}044536.1 (SEQ ID NO: 1)), glycoprotein G (gG) (e.g. NP_{—}044534.1), glycoprotein H (gH) (e.g. YP_{—}081523: NP_{—}044491.1), or glycoprotein L (gL) (e.g. YP_{—}081555: NP_{—}044470.1), UL128 (e.g. ACZ72859), UL130 (e.g. YP_{—}081565.1), or UL131a (e.g. YP_{—}081566), including mutants and variants thereof which retain antigenicity. In embodiments, a glycoprotein may be derived from an HHV as described herein, including embodiments thereof. A glycoprotein may be derived from HHV-1 or HHV-2 as described herein. Thus, in embodiments, the glycoprotein is an “HHV-1 glycoprotein” or an “HHV-2 glycoprotein”. A glycoprotein may be derived from a HSV described herein. Thus, in embodiments, the glycoprotein is a “HSV glycoprotein”. A glycoprotein may be derived from HHV-5 as described herein. Thus, in embodiments, the glycoprotein is an “HHV-5 glycoprotein”. A glycoprotein may be derived from HCMV as described herein. Thus, in embodiments, the glycoprotein is a “HCMV glycoprotein”. A glycoprotein may be derived from HHV-3, HHV-4, HHV-6, HHV-7, or HHV-8. Thus, in embodiments, a glycoprotein may be an “HHV-3 glycoprotein,” an “HHV-4 glycoprotein,” an “HHV-6 glycoprotein,” an “HHV-7 glycoprotein,” or an “HHV-8 glycoprotein”. In embodiments, the envelope protein is a component of a pentameric complex including gH, gL, UL128, UL130, and UL131A. In embodiments, the glycoprotein is glycoprotein D2 (gD2). gD2 may be derived from HSV. In embodiments the glycoprotein is glycoprotein B (gB). gB may be derived from HSV or CMV.

“Glycoprotein D2” and “gD2” is used according to its common, ordinary meaning and refers to proteins of the same or similar names and functional fragments and homologs thereof. Thus, in embodiments, gD2 may be derived from an HHV as described herein (e.g. HHV-1, HHV-2). gD2 may be derived from HHV-1 or HHV-2. In embodiments, gD2 is an “HHV-1 gD2” or an “HHV-2 gD2,”. In embodiments, gD2 is a “HSV gD2”. The term includes recombinant or naturally occurring forms of gD2 (NP_{—}044536.1: SEQ ID NO: 1), or variants thereof that maintain gD2 antigenicity (e.g. within at least 30%, 40%, 50%, 60%, 70%, 80%, 90%, 95%, or 100% as compared to SEQ ID NO: 1). The term includes recombinant or naturally occurring forms of gD2 or variants thereof that have sequence identity to SEQ ID NO: 1 (e.g. about 65%, 70%, 75%, 80%, 85%, 90%, 95%, 99% or 100% identity to SEQ ID NO: 1). In embodiments, gD2 refers to variants having N-terminal truncations of SEQ ID NO: 1. gD2 may refer to variants have mutated amino acid residues that modulate (e.g. increase or decrease) antigenicity, activity, expression, cellular targeting or protein translocation, or gD2 expression.

“Glycoprotein B” and “gB” is used according to its common, ordinary meaning and refers to proteins of the same or similar names and functional fragments and homologs thereof. Thus, in embodiments, gB may be derived from an HHV as described herein (e.g. HHV-1, HHV-2, HHV-3, HHV-4, HHV-5, HHV-6, HHV-7, or HHV-8). gB may be derived from HHV-1 (“gB1”), HHV-2 (“gB2), or HHV-5 (“gB5”). In embodiments, gB is a gB1 and/or gB2. In embodiments, gB is gB5. In embodiments, gB is a “HSV gB” or a “CMV gB”. The term includes recombinant or naturally occurring forms of gB (e.g. YP_{—}081514: (SEQ ID NO: 2); NP 044497.1; (SEQ ID NO: 3)), or variants thereof that maintain gB antigenicity (e.g. within at least 30%, 40%, 50%, 60%, 70%, 80%, 90%, 95%, or 100% as compared independently to SEQ ID NO: 2 or SEQ ID NO: 3). The term includes recombinant or naturally occurring forms of gB or variants thereof that have sequence identity to SEQ ID NO: 2 or SEQ ID NO: 3 (e.g. about 65%, 70%, 75%, 80%, 85%, 90%, 95%, 99% or 100% identity to SEQ ID NO: 2 or about 65%, 70%, 75%, 80%, 85%, 90%, 95%, 99% or 100% identity to SEQ ID NO: 3). gB may refer to variants have mutated amino acid residues that modulate (e.g. increase or decrease when compared to gB) antigenicity, activity, expression, cellular targeting or protein translocation, or gB expression.

In embodiments, the immediate early Herpes virus protein is ICP0 (e.g. NP_{—}044469.2 (SEQ ID NO: 4)), ICP4 (e.g. NP_{—}044530.1 (SEQ ID NO: 5)), IE1 (e.g. YP_{—}081562.1 (SEQ ID NO: 6)), or IE2 (e.g. YP_{—}081561.1 (SEQ ID NO: 7)), including mutants and variants thereof which retain antigenicity. The immediate early Herpes virus protein may be derived from HHV-1 or HHV-2 as described herein. Thus, in embodiments, the immediate early Herpes virus protein is an “HHV-1 immediate early Herpes virus protein” or an “HHV-2 immediate early Herpes virus protein”. The immediate early Herpes virus protein may be derived from HSV as described herein. Thus, in embodiments, the immediate early Herpes virus protein is a “HSV immediate early Herpes virus protein”. The immediate early Herpes virus protein may be derived from HHV-5 as described herein. Thus in embodiments, the immediate early Herpes virus protein is an “HHV-5 immediate early Herpes virus protein”. The immediate early Herpes virus protein may be derived from CMV as described herein. Thus in embodiments, the immediate early Herpes virus protein is a “CMV immediate early Herpes virus protein”. The immediate early Herpes virus protein may be derived from HHV-3, HHV-4, HHV-6, HHV-7, or HHV-8. Thus, in embodiments, the immediate early Herpes virus protein may be an “HHV-3 immediate early Herpes virus protein,” an “HHV-4 immediate early Herpes virus protein,” an “HHV-6 immediate early Herpes virus protein,” an “HHV-7 immediate early Herpes virus protein,” or an “HHV-8 immediate early Herpes virus protein”.

“ICP0” or “ubiquitin E3 ligase ICP0” is used according to its common, ordinary meaning and refers to proteins of the same or similar names and functional fragments and homologs thereof. Thus, in embodiments, ICP0, including its homologs, may be derived from an HHV as described herein (e.g. HHV-1, HHV-2, HHV-3, HHV-4, HHV-5, HHV-6, HHV-7, or HHV-8). ICP0 may be derived from HHV-1 or HHV-2. In embodiments, ICP0 is an “HHV-1 ICP0” or an “HHV-2 ICP0”. The term includes recombinant or naturally occurring forms of ICP0 (e.g. NP_{—}044469.2: SEQ ID NO: 4), or variants thereof that maintain ICP0 antigenicity (e.g. within at least 30%, 40%, 50%, 60%, 70%, 80%, 90%, 95%, or 100% as compared to SEQ ID NO: 4). The term includes recombinant or naturally occurring forms of ICP0 or variants thereof that have sequence identity to SEQ ID NO: 4 (e.g. about 65%, 70%, 75%, 80%, 85%, 90%, 95%, 99% or 100% identity to SEQ ID NO: 4). ICP0 may refer to variants have mutated amino acid residues that modulate (e.g. increased or decreased when compared to ICP0) antigenicity, activity, expression, cellular targeting or protein translocation, or ICP0 expression. ICP0 may be a “deactivated” as described herein (e.g. a “deactivated-ICP0”). ICP0 may be truncated as described herein (e.g. a “truncated-ICP0”). ICP0 may be modified as described herein (e.g. a “modified-ICP0”).

“ICP4” or “transcriptional regulator ICP4” is used according to its common, ordinary meaning and refers to proteins of the same or similar names and functional fragments and homologs thereof. Thus, in embodiments, ICP4, including its homologs, may be derived from an HHV as described herein (e.g. HHV-1, HHV-2, HHV-3, HHV-4, HHV-5, HHV-6, HHV-7, or HHV-8). ICP4 may be derived from HHV-1 or HHV-2. In embodiments, ICP4 is an “HHV-1 ICP4” or an “HHV-2 ICP4”. The term includes recombinant or naturally occurring forms of ICP4 (e.g. NP_{—}044530.1: SEQ ID NO: 5), or variants thereof that maintain ICP4 antigenicity (e.g. within at least 30%, 40%, 50%, 60%, 70%, 80%, 90%, 95%, or 100% as compared to SEQ ID NO: 5). The term includes recombinant or naturally occurring forms of ICP4 or variants thereof that have sequence identity to SEQ ID NO: 5 (e.g. about 65%, 70%, 75%, 80%, 85%, 90%, 95%, 99% or 100% identity to SEQ ID NO: 5). ICP4 may refer to variants have mutated amino acid residues that modulate (e.g. increased or decreased when compared to ICP4) antigenicity, activity, expression, cellular targeting or protein translocation, or ICP4 expression. ICP4 may be a “deactivated” as described herein (e.g. a “deactivated-ICP4”). ICP4 may be truncated as described herein (e.g. a “truncated-ICP4”). ICP4 may be modified as described herein (e.g. a “modified-ICP4”).

“IE1” or “regulatory protein IE1” is used according to its common, ordinary meaning and refers to proteins of the same or similar names and functional fragments and homologs thereof. Thus, in embodiments, IE1, including its homologs, may be derived from an HHV as described herein (e.g. HHV-1, HHV-2, HHV-3, HHV-4, HHV-5, HHV-6, HHV-7, or HHV-8). IE1 may be derived from HHV-5. In embodiments, IE1 is an “HHV-5 IE1”. The term includes recombinant or naturally occurring forms of IE1 (e.g. YP_{—}081562.1: SEQ ID NO: 6), or variants thereof that maintain IE1 antigenicity (e.g. within at least 30%, 40%, 50%, 60%, 70%, 80%, 90%, 95%, or 100% as compared to SEQ ID NO: 6). The term includes recombinant or naturally occurring forms of IE1 or variants thereof that have sequence identity to SEQ ID NO: 6 (e.g. about 65%, 70%, 75%, 80%, 85%, 90%, 95%, 99% or 100% identity to SEQ ID NO: 6). IE1 may refer to variants have mutated amino acid residues that modulate (e.g. increased or decreased when compared to IE1) antigenicity, activity, expression, cellular targeting or protein translocation, or IE1 expression. IE1 may be a “deactivated” as described herein (e.g. a “deactivated-IE1”). IE1 may be truncated as described herein (e.g. a “truncated-IE1”). IE1 may be modified as described herein (e.g. a “modified-IE1”).

“IE2” or “regulatory protein IE2” is used according to its common, ordinary meaning and refers to proteins of the same or similar names and functional fragments and homologs thereof. Thus, in embodiments, IE2, including its homologs, may be derived from an HHV as described herein (e.g. HHV-1, HHV-2, HHV-3, HHV-4, HHV-5, HHV-6, HHV-7, or HHV-8). IE2 may be derived from HHV-5. In embodiments, IE2 is an “HHV-5 IE2”. The term includes recombinant or naturally occurring forms of IE2 (e.g. YP_{—}081561.1: SEQ ID NO: 7), or variants thereof that maintain IE2 antigenicity (e.g. within at least 30%, 40%, 50%, 60%, 70%, 80%, 90%, 95%, or 100% as compared to SEQ ID NO: 7). The term includes recombinant or naturally occurring forms of IE2 or variants thereof that have sequence identity to SEQ ID NO: 7 (e.g. about 65%, 70%, 75%, 80%, 85%, 90%, 95%, 99% or 100% identity to SEQ ID NO: 7). IE2 may refer to variants have mutated amino acid residues that modulate (e.g. increased or decreased when compared to IE2) antigenicity, activity, expression, cellular targeting or protein translocation, or IE2 expression. IE2 may be a “deactivated” as described herein (e.g. a “deactivated-IE2”). IE1 may be truncated as described herein (e.g. a “truncated-IE2”). IE1 may be modified as described herein (e.g. a “modified-IE2”).

In embodiments, the matrix protein is UL32 (e.g. YP_{—}081491), UL83 (e.g. YP_{—}081531), UL48 (e.g. YP_{—}081506), UL46 (e.g. NP_{—}044516.1), or UL47 (e.g. NP_{—}04517.1). The matrix protein may be derived from HHV-1 or HHV-2 as described herein. Thus, in embodiments, the matrix protein is an “HHV-1 matrix protein” or an “HHV-2 matrix protein”. The matrix protein may be derived from HSV as described herein. Thus, in embodiments, the matrix is a “HSV matrix protein”. The matrix protein may be derived from HHV-5 as described herein. Thus, in embodiments, the matrix protein is an “HHV-5 matrix protein”. The matrix protein may be derived from CMV as described herein. Thus, in embodiments, the matrix is a “CMV matrix protein”. The matrix protein may be derived from HHV-3, HHV-4, HHV-6, HHV-7, or HHV-8. Thus, in embodiments, the matrix protein may be an “HHV-3 matrix protein,” an “HHV-4 matrix protein,” an “HHV-6 matrix protein,” an “HHV-7 matrix protein,” or an “HHV-8 matrix protein”.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In embodiments, the nucleic acids herein contain phosphodiester bonds. In embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

Nucleic acid is “operably linked” when it is placed into a functional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, then synthetic oligonucleotide adaptors or linkers are used in accordance with conventional practice.

The term “expression” includes any step involved in the production of the polypeptide including, but not limited to, transcription, post-transcriptional modification, translation, post-translational modification, and secretion.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to an N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “conservative substitution” as used with respect to amino acids, refers to the substitution of an amino acid with a chemically similar amino acid. Amino acid substitutions which often preserve the structural and/or functional properties of the polypeptide in which the substitution is made are known in the art and are described, for example, by H. Neurath and R. L. Hill, 1979, in “The Proteins,” Academic Press, New York. The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions. In some embodiments, there may be 1, 2, 3, 4, 5, 6, 7, 8, 9, 10, 15, 20, 25, 30, 35, or 40 conservative substitutions.

The term “amino acid substitution set” or “substitution set” refers to a group of amino acid substitutions. A substitution set can have 1, 2, 3, 4, 5, 6, 7, 8, 9, 10, 11, 12, 13, 14, 15, or more amino acid substitutions. A “point mutation” refers to mutation of a single residue in a polynucleotide or polypeptide sequence.

The term “isolated” refers to a nucleic acid, polynucleotide, polypeptide, protein, or other component that is partially or completely separated from components with which it is normally associated (other proteins, nucleic acids, cells, etc.). In embodiments, an isolated polypeptide or protein is a recombinant polypeptide or protein.

An amino acid or peptide is “heterologous” to another sequence with which it is operably linked if the two sequences are not associated in nature.

“Identity” or “percent identity,” in the context of two or more polynucleotide sequences or polypeptide sequences, refers to two or more sequences or subsequences that are the same or have a specified percentage of amino acid residues that are the same (e.g., share at least about 70%, at least about 75%, at least about 80%, at least about 85%, at least about 88% identity, at least about 89%, at least about 90%, at least about 91%, at least about 92%, at least about 93%, at least about 94%, at least about 95%, at least about 96%, at least about 97%, at least about 98%, or at least about 99% identity) over a specified region to a reference sequence, when compared and aligned for maximum correspondence over a comparison window, or designated region as measured using a sequence comparison algorithms or by manual alignment and visual inspection.

Optimal alignment of sequences for comparison and determination of sequence identity can be determined by a sequence comparison algorithm or by visual inspection (see, generally, Ausubel et al., infra). When optimally aligning sequences and determining sequence identity by visual inspection, percent sequence identity is calculated as the number of residues of the test sequence that are identical to the reference sequence divided by the number of non-gap positions and multiplied by 100. When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters as known in the art, for example BLAST or BLAST 2.0. For example, comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, or by computerized implementations of these algorithms (GAP, BESTFIT, FASTA, and TFASTA in the Wisconsin Genetics Software Package, Genetics Computer Group, 575 Science Dr., Madison, Wis.). Thus alignment can be carried out for sequences that have deletions and/or additions, as well as those that have substitutions, as well as naturally occurring, e.g., polymorphic or allelic variants, and man-made variants.

The phrase “substantial sequence identity” or “substantial identity,” in the context of two polypeptide sequences, refers to a sequence that has at least 70% identity to a reference sequence. Percent identity can be any integer from 70% to 100%. Two polypeptide sequences that have 100% sequence identity are said to be “identical.” A polypeptide sequence is said to have “substantial sequence identity” to a reference sequence when the sequences have at least about 70%, at least about 75%, at least 80%, at least 85%, at least 90%, at least 91%, at least 92%, at least 93%, at least 94%, at least 95%, at least 96%, at least 97%, at least 98%, or at least 99% or greater sequence identity as determined using the methods described herein, such as BLAST using standard parameters as described above.

The term “sulfated polysaccharide” is used according to its plain ordinary meaning in biochemistry and glycobiology and refers to two or more covalently bonded monosaccharides (including nitrogen containing monosaccharides), wherein one or more of the monosaccharides is sulfated. In embodiments, a sulfated polysaccharide may be a synthetically made or modified sulfated polysaccharide or a synthetically sulfated polysaccharide. Examples include, but are not limited to, heparin, heparan sulfate, and dextran sulfate.

The term “sulfonated polysaccharide” is used according to its plain ordinary meaning in biochemistry and glycobiology and refers to two or more covalently bonded monosaccharides (including nitrogen containing monosaccharides), wherein one or more of the monosaccharides is sulfonated. In embodiments, a sulfonated polysaccharide may be a synthetically made or modified sulfonated polysaccharide or a synthetically sulfonated polysaccharide.

“Patient” or “subject in need thereof” refers to a living organism suffering from or prone to a disease or condition that can be treated by administration of the vaccine compositions described herein. Non-limiting examples include humans, other mammals, bovines, rats, mice, dogs, monkeys, goat, sheep, cows, deer, and other non-mammalian animals. In some embodiments, a patient is human. In some embodiments, a patient or subject in need thereof or a patient in need thereof, refers to a living organism (e.g. human) at risk of developing, contracting, or having a disease or condition (e.g. HV (e.g. an HV such as, for example, HHV-1, HHV-2, HHV-3, HHV-4, HHV-5, HHV-6, HHV-7, and HHV-8) or infection or disease associated with an HV (e.g. an HV such as, for example, HHV-1, HHV-2, HHV-3, HHV-4, HHV-5, HHV-6, HHV-7, and HHV-8)).

“Disease” or “condition” refer to a state of being or health status of a patient or subject capable of being treated with the vaccine compositions or methods described herein. In embodiments, the disease is a disease related to (e.g. caused by or characterized by) HV (e.g. an HV such as, for example, HHV-1, HHV-2, HHV-3, HHV-4, HHV-5, HHV-6, HHV-7, and HHV-8). Examples of diseases, disorders, or conditions include, but are not limited to herpetic gingivostomatitis, herpes labialis, herpes genitalis, herpetic whitlow, herpes gladiatorum, herpesviral encephalitis, herpesviral meningitis, herpes esophagitis, herpes keratitis, Bell's palsy, Mollaret's meningitis, herpes rugbeiorum, eczema herpeticum, herpetic neuralgia, post-herpetic neuralgia, or disseminated neonatal herpes infection. In some instances, “disease” or “condition” refers to HV (e.g. an HV selected from the group consisting of HHV-1, HHV-2, HHV-3, HHV-4, HHV-5, HHV-6, HHV-7, and HHV-8) infection. Thus, in embodiments, the disease is caused by or characterized by HHV-5 or HCMV infection. “Disease” or “condition” may refer to herpetic gingivostomatitis. “Disease” or “condition” may refer to herpes labialis. “Disease” or “condition” may refer to herpes genitalis. “Disease” or “condition” may refer to herpetic whitlow. “Disease” or “condition” may refer to herpes gladiatorum. “Disease” or “condition” may refer to herpesviral encephalitis. “Disease” or “condition” may refer to herpesviral meningitis. “Disease” or “condition” may refer to herpes esophagitis. “Disease” or “condition” may refer to herpes keratitis. “Disease” or “condition” may refer to Bell's palsy. “Disease” or “condition” may refer to Mollaret's meningitis. “Disease” or “condition” may refer to herpes rugbeiorum. “Disease” or “condition” may refer to eczema herpeticum. “Disease” or “condition” may refer to herpetic neuralgia. “Disease” or “condition” may refer to post-herpetic neuralgia. Disease” or “condition” may refer to disseminated neonatal herpes infection. “Disease” or “condition” may refer to HSV1 infection. “Disease” or “condition” may refer to HSV2 infection. “Disease” or “condition” may refer to HSV1 and HSV2 infection. “Disease” or “condition” may refer to HHV-3 infection. “Disease” or “condition” may refer to HHV-4 infection. “Disease” or “condition” may refer to HHV-5 (e.g. HCMV) infection. “Disease” or “condition” may refer to HHV-6 infection. “Disease” or “condition” may refer to HHV-7 infection. “Disease” or “condition” may refer to HHV-8 infection. In some instances, “disease” or “condition” refers to an HV (e.g. HHV-1, HHV-2, HHV-3, HHV-4, HHV-5, HHV-6, HHV-7, or HHV-8) infection.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat HV (e.g. an HV such as, for example, HHV-1, HHV-2, HHV-3, HHV-4, HHV-5, HHV-6, HHV-7, and HHV-8) infection by decreasing the incidence of HV (e.g. an HV such as, for example, HHV-1, HHV-2, HHV-3, HHV-4, HHV-5, HHV-6, HHV-7, and HHV-8) infection, reducing one or more symptoms of HV (e.g. an HV such as, for example, HHV-1, HHV-2, HHV-3, HHV-4, HHV-5, HHV-6, HHV-7, and HHV-8) infection, or preventing the spread of HV (e.g. an HV such as, for example, HHV-1, HHV-2, HHV-3, HHV-4, HHV-5, HHV-6, HHV-7, and HHV-8). In embodiments of the vaccine compositions or methods described herein, treating HV (e.g. an HV such as, for example, HHV-1, HHV-2, HHV-3, HHV-4, HHV-5, HHV-6, HHV-7, and HHV-8) infection includes slowing the rate of growth or spread of HV (e.g. an HV such as, for example, HHV-1, HHV-2, HHV-3, HHV-4, HHV-5, HHV-6, HHV-7, and HHV-8) or reducing the occurrence of lesions. The term “treating” and conjugations thereof, include prevention of an injury, pathology, condition, or disease. The term “preventing” or “prevention” refers to any indicia of success in protecting a subject or patient (e.g. a subject or patient at risk of developing a disease or condition) from developing, contracting, or having a disease or condition (e.g. HV (e.g. an HV selected from the group consisting of HHV-1, HHV-2, HHV-3, HHV-4, HHV-5, HHV-6, HHV-7, and HHV-8) infection), including preventing one or more symptoms of a disease or condition or diminishing the occurrence, severity, or duration of any symptoms of a disease or condition following administration of a prophylactic or preventative composition as described herein.

An “effective amount” is an amount sufficient for a compound to accomplish a stated purpose relative to the absence of the compound (e.g. achieve the effect for which it is administered, treat a disease, reduce spread of HV (e.g. an HV such as, for example, HHV-1, HHV-2, HHV-3, HHV-4, HHV-5, HHV-6, HHV-7, and HHV-8), reduce one or more symptoms of a disease or condition (e.g. lesions, virus production, lytic cycle)). An example of an “effective amount” is an amount sufficient to contribute to the treatment, prevention, or reduction of a symptom or symptoms of a disease, also be referred to herein as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a composition (vaccine) is an amount of a composition that, when administered to a subject, will have the intended prophylactic effect, e.g., preventing or delaying the onset (or reoccurrence) of an injury, disease (e.g. HV (e.g. an HV such as, for example, HHV-1, HHV-2, HHV-3, HHV-4, HHV-5, HHV-6, HHV-7, and HHV-8) infection), pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses (e.g. prime-boost). Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal, intradermal, mucosal, intrarectal, intravaginal, topical, transcutaneous (e.g. as in Combadie're, PLoS ONE 5(5): e10818), or subcutaneous administration, or the implantation of a slow-release device, e.g., a mini-osmotic pump, to a subject. In embodiments, the administration is intramuscular or subcutaneous administration. Administration may be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies, for example HV (e.g. HHV-1, HHV-2, HHV-3, HHV-4, HHV-5, HHV-6, HHV-7, or HHV-8) infection therapies such as antiviral drugs (e.g. acyclovir, famciclovir, valacyclovir) or a different HV (e.g. HHV-1, HHV-2, HHV-3, HHV-4, HHV-5, HHV-6, HHV-7, or HHV-8) vaccine (e.g. DNA vaccine, DNA vaccine including different genes, isolated protein vaccine, different inactivated virus vaccine). The vaccine compositions herein can be administered alone or can be coadministered to the patient. Coadministration is meant to include simultaneous or sequential administration of the compounds individually or in combination (more than one composition) and includes vaccine administration in a prime-boost method. Thus, the preparations can also be combined, when desired, with other active substances (e.g. to reduce metabolic degradation, increase immune response (e.g. adjuvant)). The compositions described herein can be delivered transdermally, by a topical route, transcutaneously, formulated as applicator sticks, solutions, suspensions, emulsions, gels, creams, ointments, pastes, jellies, paints, powders, and aerosols. The vaccine compositions herein can be delivered intramuscularly or subcutaneously via administration of an HV vaccine. An “administration device” as used herein refers to a device used to administer the vaccine compositions described herein. Administration devices include, but are not limited, to devices for oral, topical, inhalation, or injection of the vaccine compositions described herein. Exemplary administration devices include, but are not limited to, ampoules, droppers, patches, sprays, pumps, IV lines, syringes.

The term “administer (or administering) an HV vaccine” means administering a composition that prevents or treats an HV (e.g. HHV-1, HHV-2, HHV-3, HHV-4, HHV-5, HHV-6, HHV-7, or HHV-8) infection in a subject. Administration may include, without being limited by mechanism, allowing sufficient time for the HV (e.g. HHV-1, HHV-2, HHV-3, HHV-4, HHV-5, HHV-6, HHV-7, or HHV-8) vaccine to induce an immune response in the subject or to reduce one or more symptoms of a disease (e.g. HHV-1, HHV-2, HHV-3, HHV-4, HHV-5, HHV-6, HHV-7, or HHV-8 infection).

The terms “dose” and “dosage” are used interchangeably herein. A dose refers to the amount of active ingredient given to an individual at each administration. For the present methods and compositions provided herein, the dose may generally refer to the amount of disease (e.g. HV (e.g. HHV-1, HHV-2, HHV-3, HHV-4, HHV-5, HHV-6, HHV-7, or HHV-8) infection) treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The term “vaccinate”, or additional verb forms thereof, refers to administering a vaccine to a subject (e.g. human) and eliciting an antigen specific immune response, wherein the antigen is included in the vaccine. The term “vaccinate” may also refer to eliciting an antigen specific immune response against an administered antigen. In some embodiments, vaccinate is to provide prophylaxis against a disease or infectious agent (e.g. HV, HSV, HHV-1, HHV-2, HHV-3, HHV-4, HHV-5, HHV-6, HHV-7, or HHV-8).

The term “virus particle” is used according to its plain ordinary meaning within virology and refers to a virion including the viral genome (e.g. DNA, RNA, single strand, double strand), viral capsid and associated proteins, and in the case of enveloped viruses (e.g. HV, HSV, HHV-1, HHV-2, HHV-3, HHV-4, HHV-5, HHV-6, HHV-7, or HHV-8), an envelope including lipids and optionally components of host cell membranes, and/or viral proteins.

The term “plaque forming units” is used according to its plain ordinary meaning in virology and refers to a unit of measurement based on the number of plaques per unit volume of a sample. In embodiments the units are based on the number of plaques that could form when infecting a monolayer of susceptible cells. Plaque forming unit equivalents are units of measure of inactivated virus. In embodiments, plaque forming unit equivalents are derived from plaque forming units for a sample prior to inactivation.

The term “viral shedding” is used according to its plain ordinary meaning in medicine and virology and refers to the production and release of virus from an infected cell. In embodiments, the virus is released from a cell of a subject. In embodiments virus is released into the environment from an infected subject.

The term “lesion” is used according to its plain ordinary meaning within medicine and refers to an abnormality or damage to the tissue of a subject.

“Contacting” is used in accordance with its plain ordinary meaning and refers to the process of allowing at least two distinct species (e.g. compositions, vaccines, virus, biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture. In embodiments, contacting refers to allowing radiation (e.g. UV, gamma) to interact with matter (e.g. virus, virus component, virus protein, virus nucleic acid). In embodiments, contacting refers to allowing the vaccine compositions described herein to interact with a host immune system to elicit an immunological response.

The term “contacting” may include allowing two species to react, interact, or physically touch, wherein the two species may be a composition (e.g. vaccine) as described herein and a cell, virus, virus particle, protein, enzyme, or patient. In embodiments contacting includes allowing a composition described herein to interact with a protein or enzyme that is involved in a signaling pathway. In embodiments contacting includes allowing a composition described herein to interact with an HV (e.g. an HV selected from the group consisting of HHV-1, HHV-2, HHV-3, HHV-4, HHV-5, HHV-6, HHV-7, and HHV-8). In embodiments contacting includes allowing an agent described herein to interact with an HV (e.g. an HV selected from the group consisting of HHV-1, HHV-2, HHV-3, HHV-4, HHV-5, HHV-6, HHV-7, and HHV-8) and inactivate the HV (e.g. an HV selected from the group consisting of HHV-1, HHV-2, HHV-3, HHV-4, HHV-5, HHV-6, HHV-7, and HHV-8).

The term “associated” or “associated with” as used herein to describe a disease (e.g. a virus associated disease, HV associated disease, HSV associated disease, HHV-1 associated disease, HHV-2 associated disease, HHV-3 associated disease, HHV-4 associated disease, HHV-5 associated disease, HHV-6 associated disease, HHV-7 associated disease, or HHV-8 associated disease) refers to a disease caused by, characterized by, or a symptom of the disease is caused or characterized by, what is described as disease associated or what is described as associated with the disease. For example, a disease associated with HSV2 or HSV2 infection may be a disease that results (entirely or partially) from HSV2 or HSV2 infection. As used herein, what is described as being associated with a disease, if a causative agent, could be a target for treatment of the disease. For example, a disease associated with HSV2 or HSV2 infection, may be treated with an HSV2 vaccine as described herein (including embodiments). For example, a disease associated with HSV2 may be a disease that a subject with HSV2 or HSV2 infection is at higher risk of developing as compared to a subject without HSV2 or HSV2 infection.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of HV (e.g. an HV selected from the group consisting of HHV-1, HHV-2, HHV-3, HHV-4, HHV-5, HHV-6, HHV-7, and HHV-8) infection or one or more symptoms of HV (e.g. an HV selected from the group consisting of HHV-1, HHV-2, HHV-3, HHV-4, HHV-5, HHV-6, HHV-7, and HHV-8) infection in the absence of a composition (e.g. vaccine) as described herein (including embodiments).

### I. Compositions

Provided herein are vaccine compositions and methods of using the same in treating a Herpes disease in a subject in need thereof. The vaccine compositions herein include an inactivated Herpes virus, a recombinant-Herpes virus protein, and a vaccine adjuvant.

In embodiments, the inactivated Herpes virus is an HHV-1, HHV-2, HHV-3, HHV-4, HHV-5, HHV-6, HHV-7, or HHV-8 Herpes virus. The inactivated Herpes virus may be HHV-1, HHV-2, or HHV-5. The inactivated Herpes virus may be HHV-1 or HHV-2. The inactivated Herpes virus may be HHV-1. The inactivated Herpes virus may be HHV-2. The inactivated Herpes virus may be HSV-1 or HSV-2. The inactivated Herpes virus may be HSV-1. The inactivated Herpes virus may be HSV-2. The inactivated Herpes virus may be HHV-5. The inactivated Herpes virus may be HCMV. The vaccine composition may include a single inactivated Herpes virus (e.g. a monovalent vaccine composition). The monovalent vaccine composition may include HSV-1, HSV-2, or HCMV. The monovalent vaccine composition may include HSV-1 or HSV-2. The monovalent vaccine composition may include HCMV. The monovalent vaccine composition may include HSV-1. The monovalent vaccine composition may include HSV-2. In embodiments, the vaccine composition includes two or more inactivated Herpes viruses. In embodiments, the vaccine composition includes two or more inactivated Herpes viruses where at least two of the Herpes viruses are HSV-1 or HSV-2. In embodiments, the vaccine composition includes two inactivated Herpes viruses (e.g. a divalent vaccine composition). The divalent vaccine composition may include HSV-1 and HSV-2.

In embodiments the inactivated Herpes virus is formed by chemical inactivation. The chemical agent may be aziridine. The chemical agent may be ethylenimine. (e.g. binary ethylenimine). The chemical agent may be beta-propiolactone. The chemical agent may be formaldehyde. The chemical agent may be formalin. The chemical agent may be sodium periodate. The chemical agent may be hydrogen peroxide. The chemical agent may be aldrithiol-2. The chemical agent may be Triton-X-100. The chemical agent may be NP-40. The chemical agent may be Tween-20. In embodiments, the chemical agent is furocoumarin. The furocoumarin may be psoralen, 4′-aminomethyl-4, 5′, 8-trimethylpsoralen, angelicin, xanthotoxin, bergapten, or nodakenetin. In embodiments, the inactivated Herpes virus is inactivated by contacting Herpes virus with formalin (e.g. a “formalin-inactivated HV” or “FI-HV”) Thus, the FI-HV may be a FI-HHV-1, FI-HSV-1, FI-HHV-2, or FI-HSV-2. The FI-HV may be a FI-HHV-5 or FI-HCMV. In embodiments, the inactivated Herpes virus is formed by contacting with radiation. The radiation may be UV radiation, electron beam radiation, infrared radiation, or gamma radiation.

In embodiments the inactivated Herpes virus is formed by contacting the Herpes virus with one or more agents such as heat, radiation, or a chemical agent. Thus, in embodiments, the inactivated Herpes virus is formed by contacting radiation as described herein and chemical inactivation such as contact with formalin.

In embodiments the inactivated Herpes virus is an inactivated single strain of the Herpes virus. The inactivated Herpes virus may be a combination of two or more inactivated strains of the HV. The inactivated Herpes virus may be an inactivated single strain of HSV1. The inactivated Herpes virus may be an inactivated single strain of HSV2. The inactivated Herpes virus may be a combination of two or more inactivated strains of HSV1. The inactivated Herpes virus may be a combination of two or more inactivated strains of HSV2. The inactivated Herpes virus may be a combination of one or more inactivated strains or HSV1 and one or more inactivated strains of HSV2.

The vaccine adjuvant may be a lipopolysaccharide (LPS)-adjuvant. The LPS-adjuvant is as described herein, including embodiments thereof. Thus, in embodiments, the LPS-adjuvant is monophosphoryl lipid A (MPL). The adjuvant may be an aluminum-based mineral salt adjuvant as described herein, including embodiments thereof. Thus, in embodiments, the aluminum-based mineral salt adjuvant is aluminum hydroxide or aluminum phosphate. The aluminum-based mineral salt adjuvant may be aluminum hydroxide, including embodiments described herein. The aluminum-based mineral salt adjuvant may be aluminum phosphate, including embodiments described herein. The aluminum-based mineral salt adjuvant may be Alhydrogel™ or Adju-phos™ In embodiments, an aluminum-based mineral salt adjuvant is not Adju-phos™. In embodiments, the aluminum-based mineral salt adjuvant is a potassium aluminum sulfate adjuvant as described herein.

In embodiments, the LPS-adjuvant is derived from the Salmonella minnesota LPS. The lipopolysaccharide-adjuvant may be derived from the Salmonella minnesota Re595 LPS. The lipopolysaccharide-adjuvant may be derived from the R595 LPS. The lipopolysaccharide-adjuvant may be a lipid A adjuvant. The lipopolysaccharide-adjuvant may be a lipid A adjuvant without an (R)-3-hydroxytetradecanoyl moiety. The lipopolysaccharide-adjuvant may be a lipid A adjuvant without a 1-phosphate moiety. The lipopolysaccharide-adjuvant may be 3-O-desacyl-4′-monophosphoryl lipid A. The lipopolysaccharide-adjuvant may be capable of binding the TLR4 protein. The lipopolysaccharide-adjuvant may be a synthetic MPL analogue adjuvant.

In embodiments the aluminum-based mineral salt adjuvant is aluminum hydroxide adjuvant. The aluminum-based mineral salt adjuvant may be an aluminum phosphate adjuvant. The aluminum-based mineral salt adjuvant may be a potassium aluminum sulfate adjuvant. The aluminum-based mineral salt adjuvant may include crystalline aluminum hydroxide and not amorphous aluminum hydroxide or aluminum hydroxycarbonate or magnesium hydroxide. The aluminum-based mineral salt adjuvant may include aluminum phosphate gel in the form of a white gelatinous precipitate. The aluminum-based mineral salt adjuvant may be include aluminum hydroxide gel in the form of a white gelatinous precipitate.

In embodiments, the vaccine adjuvant includes two or more adjuvants. Thus, in embodiments, the vaccine adjuvant includes one or more aluminum-based mineral salt adjuvants as described herein, including embodiments thereof. The vaccine adjuvant may include one or more aluminum-based mineral salt adjuvant and a LPS-adjuvant. Thus, in embodiments, the vaccine adjuvant may include MPL and aluminum hydroxide or aluminum phosphate. The vaccine adjuvant may include MPL and Alhydrogel™. In embodiments, the vaccine adjuvant includes MPL and an aluminum-based mineral salt adjuvant other than Adju-phos™.

In embodiments, the recombinant-Herpes virus protein is a full-length recombinant-Herpes virus protein. The recombinant-Herpes virus protein may be a fragment of a full-length recombinant-Herpes virus protein, which retains antigenicity, and may be, for example, about 10, 20, 50, 100, 150, 200, 250, 300, 350, 400, 450, 500, 550, 600, 650, 700, 750, 800, 850, 900, 950, 1000, 1100, 1200, 1300, 1400, or 1500 amino acids of a full-length recombinant-Herpes virus protein.

In embodiments, the recombinant-Herpes virus protein present at an amount about 2-fold to about 100-fold greater than its corresponding endogenous-Herpes virus protein. In embodiments, the recombinant-Herpes virus protein present at an amount about 5-fold to about 100-fold greater than its corresponding endogenous-Herpes virus protein. In embodiments, the recombinant-Herpes virus protein present at an amount about 10-fold to about 100-fold greater than its corresponding endogenous-Herpes virus protein. In embodiments, the recombinant-Herpes virus protein present at an amount about 20-fold to about 100-fold greater than its corresponding endogenous-Herpes virus protein. In embodiments, the recombinant-Herpes virus protein present at an amount about 30-fold to about 100-fold greater than its corresponding endogenous-Herpes virus protein. In embodiments, the recombinant-Herpes virus protein present at an amount about 40-fold to about 100-fold greater than its corresponding endogenous-Herpes virus protein. In embodiments, the recombinant-Herpes virus protein present at an amount about 50-fold to about 100-fold greater than its corresponding endogenous-Herpes virus protein. In embodiments, the recombinant-Herpes virus protein present at an amount about 60-fold to about 100-fold greater than its corresponding endogenous-Herpes virus protein. In embodiments, the recombinant-Herpes virus protein present at an amount about 70-fold to about 100-fold greater than its corresponding endogenous-Herpes virus protein. In embodiments, the recombinant-Herpes virus protein present at an amount about 80-fold to about 100-fold greater than its corresponding endogenous-Herpes virus protein. In embodiments, the recombinant-Herpes virus protein present at an amount about 90-fold to about 100-fold greater than its corresponding endogenous-Herpes virus protein.

In embodiments, the recombinant-Herpes virus protein present at an amount about 2-fold to about 90-fold greater than its corresponding endogenous-Herpes virus protein. In embodiments, the recombinant-Herpes virus protein present at an amount about 2-fold to about 80-fold greater than its corresponding endogenous-Herpes virus protein. In embodiments, the recombinant-Herpes virus protein present at an amount about 2-fold to about 70-fold greater than its corresponding endogenous-Herpes virus protein. In embodiments, the recombinant-Herpes virus protein present at an amount about 2-fold to about 60-fold greater than its corresponding endogenous-Herpes virus protein. In embodiments, the recombinant-Herpes virus protein present at an amount about 2-fold to about 50-fold greater than its corresponding endogenous-Herpes virus protein. In embodiments, the recombinant-Herpes virus protein present at an amount about 2-fold to about 40-fold greater than its corresponding endogenous-Herpes virus protein. In embodiments, the recombinant-Herpes virus protein present at an amount about 2-fold to about 30-fold greater than its corresponding endogenous-Herpes virus protein. In embodiments, the recombinant-Herpes virus protein present at an amount about 2-fold to about 20-fold greater than its corresponding endogenous-Herpes virus protein. In embodiments, the recombinant-Herpes virus protein present at an amount about 2-fold to about 10-fold greater than its corresponding endogenous-Herpes virus protein. In embodiments, the recombinant-Herpes virus protein present at an amount about 2-fold to about 5-fold greater than its corresponding endogenous-Herpes virus protein.

In embodiments, the recombinant-Herpes virus protein present at an amount about 5-fold to about 90-fold greater than its corresponding endogenous-Herpes virus protein. In embodiments, the recombinant-Herpes virus protein present at an amount about 5-fold to about 80-fold greater than its corresponding endogenous-Herpes virus protein. In embodiments, the recombinant-Herpes virus protein present at an amount about 5-fold to about 70-fold greater than its corresponding endogenous-Herpes virus protein. In embodiments, the recombinant-Herpes virus protein present at an amount about 5-fold to about 60-fold greater than its corresponding endogenous-Herpes virus protein. In embodiments, the recombinant-Herpes virus protein present at an amount about 5-fold to about 50-fold greater than its corresponding endogenous-Herpes virus protein. In embodiments, the recombinant-Herpes virus protein present at an amount about 5-fold to about 40-fold greater than its corresponding endogenous-Herpes virus protein. In embodiments, the recombinant-Herpes virus protein present at an amount about 5-fold to about 30-fold greater than its corresponding endogenous-Herpes virus protein. In embodiments, the recombinant-Herpes virus protein present at an amount about 5-fold to about 20-fold greater than its corresponding endogenous-Herpes virus protein. In embodiments, the recombinant-Herpes virus protein present at an amount about 5-fold to about 10-fold greater than its corresponding endogenous-Herpes virus protein.

In embodiments, the recombinant-Herpes virus protein present at an amount about 10-fold to about 90-fold greater than its corresponding endogenous-Herpes virus protein. In embodiments, the recombinant-Herpes virus protein present at an amount about 10-fold to about 80-fold greater than its corresponding endogenous-Herpes virus protein. In embodiments, the recombinant-Herpes virus protein present at an amount about 10-fold to about 70-fold greater than its corresponding endogenous-Herpes virus protein. In embodiments, the recombinant-Herpes virus protein present at an amount about 10-fold to about 60-fold greater than its corresponding endogenous-Herpes virus protein. In embodiments, the recombinant-Herpes virus protein present at an amount about 10-fold to about 50-fold greater than its corresponding endogenous-Herpes virus protein. In embodiments, the recombinant-Herpes virus protein present at an amount about 10-fold to about 40-fold greater than its corresponding endogenous-Herpes virus protein. In embodiments, the recombinant-Herpes virus protein present at an amount about 10-fold to about 30-fold greater than its corresponding endogenous-Herpes virus protein. In embodiments, the recombinant-Herpes virus protein present at an amount about 10-fold to about 20-fold greater than its corresponding endogenous-Herpes virus protein.

In embodiments, the recombinant-Herpes virus protein present at an amount about 20-fold to about 90-fold greater than its corresponding endogenous-Herpes virus protein. In embodiments, the recombinant-Herpes virus protein present at an amount about 20-fold to about 80-fold greater than its corresponding endogenous-Herpes virus protein. In embodiments, the recombinant-Herpes virus protein present at an amount about 20-fold to about 70-fold greater than its corresponding endogenous-Herpes virus protein. In embodiments, the recombinant-Herpes virus protein present at an amount about 20-fold to about 60-fold greater than its corresponding endogenous-Herpes virus protein. In embodiments, the recombinant-Herpes virus protein present at an amount about 20-fold to about 50-fold greater than its corresponding endogenous-Herpes virus protein. In embodiments, the recombinant-Herpes virus protein present at an amount about 20-fold to about 40-fold greater than its corresponding endogenous-Herpes virus protein. In embodiments, the recombinant-Herpes virus protein present at an amount about 20-fold to about 30-fold greater than its corresponding endogenous-Herpes virus protein.

In embodiments, the recombinant-Herpes virus protein present at an amount about 30-fold to about 90-fold greater than its corresponding endogenous-Herpes virus protein. In embodiments, the recombinant-Herpes virus protein present at an amount about 30-fold to about 80-fold greater than its corresponding endogenous-Herpes virus protein. In embodiments, the recombinant-Herpes virus protein present at an amount about 30-fold to about 70-fold greater than its corresponding endogenous-Herpes virus protein. In embodiments, the recombinant-Herpes virus protein present at an amount about 30-fold to about 60-fold greater than its corresponding endogenous-Herpes virus protein. In embodiments, the recombinant-Herpes virus protein present at an amount about 30-fold to about 50-fold greater than its corresponding endogenous-Herpes virus protein. In embodiments, the recombinant-Herpes virus protein present at an amount about 30-fold to about 40-fold greater than its corresponding endogenous-Herpes virus protein.

In embodiments, the recombinant-Herpes virus protein present at an amount about 40-fold to about 90-fold greater than its corresponding endogenous-Herpes virus protein. In embodiments, the recombinant-Herpes virus protein present at an amount about 40-fold to about 80-fold greater than its corresponding endogenous-Herpes virus protein. In embodiments, the recombinant-Herpes virus protein present at an amount about 40-fold to about 70-fold greater than its corresponding endogenous-Herpes virus protein. In embodiments, the recombinant-Herpes virus protein present at an amount about 40-fold to about 60-fold greater than its corresponding endogenous-Herpes virus protein. In embodiments, the recombinant-Herpes virus protein present at an amount about 40-fold to about 50-fold greater than its corresponding endogenous-Herpes virus protein.

In embodiments, the recombinant-Herpes virus protein present at an amount about 50-fold to about 90-fold greater than its corresponding endogenous-Herpes virus protein. In embodiments, the recombinant-Herpes virus protein present at an amount about 50-fold to about 80-fold greater than its corresponding endogenous-Herpes virus protein. In embodiments, the recombinant-Herpes virus protein present at an amount about 50-fold to about 70-fold greater than its corresponding endogenous-Herpes virus protein. In embodiments, the recombinant-Herpes virus protein present at an amount about 50-fold to about 60-fold greater than its corresponding endogenous-Herpes virus protein.

The vaccine compositions herein may include a recombinant-Herpes virus protein present at an amount about 2-fold greater than its corresponding endogenous-Herpes virus protein. The vaccine compositions herein may include a recombinant-Herpes virus protein present at an amount about 5-fold greater than its corresponding endogenous-Herpes virus protein. The vaccine compositions herein may include a recombinant-Herpes virus protein present at an amount about 10-fold greater than its corresponding endogenous-Herpes virus protein. The vaccine compositions herein may include a recombinant-Herpes virus protein present at an amount about 20-fold greater than its corresponding endogenous-Herpes virus protein. The vaccine compositions herein may include a recombinant-Herpes virus protein present at an amount about 30-fold greater than its corresponding endogenous-Herpes virus protein. The vaccine compositions herein may include a recombinant-Herpes virus protein present at an amount about 40-fold greater than its corresponding endogenous-Herpes virus protein. The vaccine compositions herein may include a recombinant-Herpes virus protein present at an amount about 50-fold greater than its corresponding endogenous-Herpes virus protein. The vaccine compositions herein may include a recombinant-Herpes virus protein present at an amount about 60-fold greater than its corresponding endogenous-Herpes virus protein. The vaccine compositions herein may include a recombinant-Herpes virus protein present at an amount about 70-fold greater than its corresponding endogenous-Herpes virus protein. The vaccine compositions herein may include a recombinant-Herpes virus protein present at an amount about 80-fold greater than its corresponding endogenous-Herpes virus protein. The vaccine compositions herein may include a recombinant-Herpes virus protein present at an amount about 90-fold greater than its corresponding endogenous-Herpes virus protein. The vaccine compositions herein may include a recombinant-Herpes virus protein present at an amount about 100-fold greater than its corresponding endogenous-Herpes virus protein.

The recombinant-Herpes virus protein may include an antigenic site. In embodiments, the recombinant-Herpes virus protein includes two or more antigenic sites. The recombinant-Herpes virus protein may be deactivated (e.g. a “deactivated-recombinant-Herpes virus protein”). The recombinant-Herpes virus protein may be a fragment of a full-length recombinant-Herpes virus protein which includes an antigenic site. In embodiments, the recombinant-Herpes virus protein is a fragment of a full-length recombinant-Herpes virus protein that includes two or more antigenic sites. The recombinant-Herpes virus protein may be deactivated (e.g. a “deactivated-recombinant-Herpes virus protein”). The deactivated-recombinant-Herpes virus protein may include a deactivating mutation (e.g. a mutation which deactivates the recombinant-Herpes virus protein).

In embodiments, the recombinant-Herpes virus protein is an envelope protein, an immediate early Herpes virus protein, a capsid protein, a matrix protein, an early Herpes virus protein, early-late Herpes virus protein, late Herpes virus protein, structural Herpes virus protein, non-structural Herpes virus protein, or a replication Herpes virus protein, or a fragment thereof that retains antigenicity. In embodiments the recombinant-Herpes virus protein is an envelope protein, an immediate early Herpes virus protein, a capsid protein, a matrix protein, or an early Herpes virus protein, or a fragment thereof that retains antigenicity. In embodiments, the recombinant-Herpes virus protein is an envelope protein, an immediate early Herpes virus protein, a capsid protein, or a matrix protein, or a fragment thereof that retains antigenicity. In embodiments, the recombinant-Herpes virus protein is an envelope protein, an immediate early Herpes virus protein, or a capsid protein or a fragment thereof that retains antigenicity. In embodiments, the recombinant-Herpes virus protein is an envelope protein or an immediate early Herpes virus protein or a fragment thereof that retains antigenicity. In embodiments, the recombinant-Herpes virus protein is a protein, or antigenic fragment thereof as set forth in Table 1.

TABLE 1

| Protein | Accession No or SEQ ID NO: |
|---|---|
| glycoprotein B (gB) | YP_081514 (SEQ ID NO: 2): NP_044497.1 |
| | (SEQ ID NO: 3) |
| glycoprotein D (gD) | NP_044536.1 (SEQ ID NO: 1): |
| glycoprotein G (gG) | NP_044534.1: |
| glycoprotein H (gH) | YP_081523: NP_044491.1 |
| glycoprotein L (gL) | YP_081555: NP_044470.1 |
| UL128 | ACZ72859: |
| UL130 | YP_081565.1: |
| UL131a | YP_081566: |
| ICP0 | NP_044469.2: (SEQ ID NO: 4) |
| ICP4 | NP_044530.1: (SEQ ID NO: 5) |
| IE1 | YP_081562.1: (SEQ ID NO: 6) |
| IE2 | YP_081561.1: (SEQ ID NO: 7) |
| UL32 | YP_081491.1: YP_081491 |
| UL83 | ABV71605.1: YP_081531 |
| UL48 | ABV71578.1: YP_081506 |
| UL46 | NP_044516.1 |
| UL47 | NP_044517.1 |
| UL19 | NP_044488.1 |
| UL29 | NP_044499.1 |
| UL5 | NP_044474.1 |
| UL30 | NP_044500.1 |
| UL38 | NP_044508.1 |
| UL42 | NP_044512.1 |
| US9 | NP_044540.1 |

The recombinant-Herpes virus protein may be a structural protein or a non-structural protein such as HSV-1, HSV-2, UL19, UL46, for UL29 or fragment thereof which retains antigenicity. The recombinant-Herpes virus protein may be a replication protein (e.g. a DNA replication complex protein, a protein essential for replication (e.g. HSV-1, HSV-2, UL5, UL30, or UL42), or a protein dispensable for virus replication (e.g. HSV-1, HSV-2, US9) or fragment thereof which retains antigenicity. The recombinant-Herpes virus protein may be an early protein or fragment thereof which retains antigenicity. In embodiments, the early protein is HSV-1, HSV-2, UL5, UL30, or UL29. The recombinant-Herpes virus protein may be an early-late protein. In embodiments, the early-late protein is HSV-1, HSV-2, or UL19. The recombinant-Herpes virus protein may be a late protein. In embodiments, the late protein is HSV-1, HSV-2, or UL38.

The recombinant-Herpes virus protein may be a capsid protein as described herein, including embodiments thereof such as, for example, fragments thereof which retain antigenicity. The capsid protein may be HSV-1, HSV-2 UL19 or UL38 and may optionally be derived from HSV-1, HSV-2. In embodiments, the recombinant-Herpes virus protein is a matrix protein as described herein, including fragments thereof which retain antigenicity. The matrix protein may be UL46 or UL47 or fragment thereof which retains antigenicity, and may optionally be derived from HSV-1, HSV-2.

In embodiments, the capsid protein or matrix proteins is a fragment of a full-length capsid protein or matrix protein which retains antigenicity. The fragment of the capsid protein or matrix protein may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the capsid protein or matrix protein may be at least about 50 amino acids long. The fragment of the capsid protein or matrix protein may be at least about 100 amino acids long. The fragment of the capsid protein or matrix protein may be at least about 150 amino acids long. The fragment of the capsid protein or matrix protein may be at least about 200 amino acids long. The fragment of the capsid protein or matrix protein may be at least about 250 amino acids long. The fragment of the capsid protein or matrix protein may be at least about 300 amino acids long. The fragment of the capsid protein or matrix protein may be at least about 350 amino acids long. The fragment of the capsid protein or matrix protein may be at least about 400 amino acids long. The fragment of the capsid protein or matrix protein may be at least about 450 amino acids long. The fragment of the capsid protein or matrix protein may be at least about 500 amino acids long. The fragment of the capsid protein or matrix protein may be at least about 550 amino acids long. The fragment of the capsid protein or matrix protein may be at least about 600 amino acids long. The fragment of the capsid protein or matrix protein may be at least about 650 amino acids long. The fragment of the capsid protein or matrix protein may be at least about 700 amino acids long. The fragment of the capsid protein or matrix protein may be at least about 750 amino acids long. The fragment of the capsid protein or matrix protein may be at least about 800 amino acids long. The fragment of the capsid protein or matrix protein may be at least about 850 amino acids long. The fragment of the capsid protein or matrix protein may be at least about 900 amino acids long. The fragment of the capsid protein or matrix protein may be at least about 950 amino acids long. The fragment of the capsid protein or matrix protein may be at least about 1000 amino acids long. The fragment of the capsid protein or matrix protein may be at least about 1100 amino acids long. The fragment of the capsid protein or matrix protein may be at least about 1200 amino acids long. The fragment of the capsid protein or matrix protein may be at least about 1300 amino acids long. The fragment of the capsid protein or matrix protein may be at least about 1400 amino acids long. The fragment of the capsid protein or matrix protein may be at least about 1500 amino acids long.

The recombinant-Herpes virus protein may be an envelope protein as described herein or fragment thereof which retains antigenicity. The envelope protein may be derived from an HHV described herein, including embodiments thereof or fragment thereof which retains antigenicity. Thus, in embodiments, the envelope protein is an HHV-1 envelope protein, an HHV-2 envelope protein, HHV-3 envelope protein, HHV-4 envelope protein, HHV-5 envelope protein, HHV-6 envelope protein, HHV-7 envelope protein, or HHV-8 envelope protein or fragment thereof which retains antigenicity. The envelope protein may be derived from HSV (e.g. HSV-1 or HSV-2) or fragment thereof which retains antigenicity. Thus, the envelope protein may be a HSV-1 envelope protein or fragment thereof which retains antigenicity. The envelope protein may be a HSV-2 envelope protein. The envelope protein may be derived from HCMV (e.g. a HCMV envelope protein) or fragment thereof which retains antigenicity

In embodiments, the envelope protein is a fragment of a full-length envelope protein which retains antigenicity. The fragment of the envelope protein may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the envelope protein may be at least about 50 amino acids long. The fragment of the envelope protein may be at least about 100 amino acids long. The fragment of the envelope protein may be at least about 150 amino acids long. The fragment of the envelope protein may be at least about 200 amino acids long. The fragment of the envelope protein may be at least about 250 amino acids long. The fragment of the envelope protein may be at least about 300 amino acids long. The fragment of the envelope protein may be at least about 350 amino acids long. The fragment of the envelope protein may be at least about 400 amino acids long. The fragment of the envelope protein may be at least about 450 amino acids long. The fragment of the envelope protein may be at least about 500 amino acids long. The fragment of the envelope protein may be at least about 550 amino acids long. The fragment of the envelope protein may be at least about 600 amino acids long. The fragment of the envelope protein may be at least about 650 amino acids long. The fragment of the envelope protein may be at least about 700 amino acids long. The fragment of the envelope protein may be at least about 750 amino acids long. The fragment of the envelope protein may be at least about 800 amino acids long. The fragment of the envelope protein may be at least about 850 amino acids long. The fragment of the envelope protein may be at least about 900 amino acids long. The fragment of the envelope protein may be at least about 950 amino acids long. The fragment of the envelope protein may be at least about 1000 amino acids long. The fragment of the envelope protein may be at least about 1100 amino acids long. The fragment of the envelope protein may be at least about 1200 amino acids long. The fragment of the envelope protein may be at least about 1300 amino acids long. The fragment of the envelope protein may be at least about 1400 amino acids long. The fragment of the envelope protein may be at least about 1500 amino acids long.

In embodiments, the envelope protein is a glycoprotein as described herein. In embodiments, the glycoprotein is gB, gD, gG, gH, or gL. The glycoprotein may be gB. The glycoprotein may be gD. The glycoprotein may be gG. The glycoprotein may be gH. The glycoprotein may be gL. The glycoprotein may be derived from HHV-1 or HHV-2. In embodiments, the glycoprotein is derived from HHV-1. In embodiments, the glycoprotein is derived from HHV-2. In embodiments, the glycoprotein is derived from HSV (e.g. a “HSV glycoprotein” as described herein). The glycoprotein may be gD and optionally be derived from HSV (e.g. HSV-1 or HSV-2). Where the glycoprotein is gD, the gD may be glycoprotein D2 (gD2: SEQ ID NO: 1). The gD2 may include an antigenic site. In embodiments, the gD2 includes at least two antigenic sites. The gD2 may be a truncated-gD2 (e.g. “gD2t”—a gD2 with a deletion of N-terminal or C-terminal residues from SEQ ID NO: 1). In embodiments, the truncated-gD2 corresponds to deletion of about 150 amino acids from the C-terminus of SEQ ID NO: 1. In embodiments, the truncated-gD2 corresponds to deletion of about 125 amino acids from the C-terminus of SEQ ID NO: 1. In embodiments, the truncated-gD2 corresponds to deletion of about 100 amino acids from the C-terminus of SEQ ID NO: 1. In embodiments, the truncated-gD2 corresponds to deletion of about 95 amino acids from the C-terminus of SEQ ID NO: 1. In embodiments, the truncated-gD2 corresponds to deletion of about 90 amino acids from the C-terminus of SEQ ID NO: 1. In embodiments, the truncated-gD2 corresponds to deletion of about 85 amino acids from the C-terminus of SEQ ID NO: 1. In embodiments, the truncated-gD2 corresponds to deletion of about 80 amino acids from the C-terminus of SEQ ID NO: 1. In embodiments, the truncated-gD2 corresponds to deletion of about 75 amino acids from the C-terminus of SEQ ID NO: 1. In embodiments, the truncated-gD2 corresponds to deletion of about 70 amino acids from the C-terminus of SEQ ID NO: 1. In embodiments, the truncated-gD2 corresponds to deletion of about 65 amino acids from the C-terminus of SEQ ID NO: 1. In embodiments, the truncated-gD2 corresponds to deletion of about 60 amino acids from the C-terminus of SEQ ID NO: 1. In embodiments, the truncated-gD2 corresponds to deletion of about 55 amino acids from the C-terminus of SEQ ID NO: 1. In embodiments, the truncated-gD2 corresponds to deletion of about 50 amino acids from the C-terminus of SEQ ID NO: 1. In embodiments, the truncated-gD2 corresponds to deletion of about residues 328 to about 393 of SEQ ID NO: 1.

In embodiments, the gD is a fragment of a full-length gD which retains antigenicity. The fragment of the gD may be about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the gD may be about 50 amino acids long. The fragment of the gD may be about 100 amino acids long. The fragment of the gD may be about 150 amino acids long. The fragment of the gD may be about 200 amino acids long. The fragment of the gD may be about 250 amino acids long. The fragment of the gD may be about 300 amino acids long. The fragment of the gD may be about 350 amino acids long. The fragment of the gD may be about 400 amino acids long.

In embodiments, the glycoprotein is derived from HHV-5. Thus, the glycoprotein may a HCMV glycoprotein as described herein, including embodiments thereof. The glycoprotein may be gB and optionally be derived from CMV (e.g. HCMV). Where the gB is derived from HCMV, the gB (i.e. gB5) may correspond to SEQ ID NO: 2. Alternatively, the gB may be derived from HSV (e.g. HSV-1 or HSV-2). In embodiments the gB is derived from HSV (e.g. HSV-1 (“gB1”) or HSV-2 (“gB2”)). Where the gB is derived from HSV, the gB (i.e. gB1 or gB2 may correspond to SEQ ID NO: 3. The gB may include an antigenic site. In embodiments, the gB includes at least two antigenic sites. The gB may be a modified-gB, where the modified-gB includes a mutation. The mutation may be deactivating mutation. The gB may be a truncated-gB (e.g. “gBt”—a gB with a deletion of N-terminal or C-terminal residues from either SEQ ID NO: 2 or SEQ ID NO: 3). In embodiments, the glycoprotein is a component of a pentameric complex derived from CMV. The pentameric complex includes gH, gL, UL128, UL130 and UL131A as described herein, including embodiments thereof.

In embodiments, the gB is a fragment of a full-length gB which retains antigenicity. The fragment of the gB may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the gB may be at least about 50 amino acids long. The fragment of the gB may be at least about 100 amino acids long. The fragment of the gB may be at least about 150 amino acids long. The fragment of the gB may be at least about 200 amino acids long. The fragment of the gB may be at least about 250 amino acids long. The fragment of the gB may be at least about 300 amino acids long. The fragment of the gB may be at least about 350 amino acids long. The fragment of the gB may be at least about 400 amino acids long. The fragment of the gB may be at least about 450 amino acids long. The fragment of the gB may be at least about 500 amino acids long. The fragment of the gB may be at least about 550 amino acids long. The fragment of the gB may be at least about 600 amino acids long. The fragment of the gB may be at least about 650 amino acids long. The fragment of the gB may be at least about 700 amino acids long. The fragment of the gB may be at least about 750 amino acids long. The fragment of the gB may be at least about 800 amino acids long. The fragment of the gB may be at least about 850 amino acids long. The fragment of the gB may be at least about 900 amino acids long. The fragment of the gB may be at least about 925 amino acids long.

In embodiments, the recombinant-Herpes virus protein is an immediate early Herpes virus protein. The immediate early Herpes virus protein may be derived from an HHV as described herein (e.g. HHV-1, HHV-2, HHV-3, HHV-4, HHV-5, HHV-6, HHV-7, or HHV-8). In embodiments, immediate early Herpes virus protein is derived from HHV-1 or HHV-2. The immediate early Herpes virus protein may be derived from HHV-1. The immediate early Herpes virus protein may be derived from HHV-2. The immediate early Herpes virus protein may be derived from HSV (e.g. HSV-1 or HSV-2). The immediate early Herpes virus protein may be derived from HSV-1. The immediate early Herpes virus protein may be derived from HSV-2. The immediate early Herpes virus protein may be derived from HHV-5. The immediate early Herpes virus protein may be derived from HCMV.

In embodiments, the immediate early protein is a fragment of a full-length immediate early protein which retains antigenicity. The fragment of the immediate early protein may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the immediate early protein may be at least about 50 amino acids long. The fragment of the immediate early protein may be at least about 100 amino acids long. The fragment of the immediate early protein may be at least about 150 amino acids long. The fragment of the immediate early protein may be at least about 200 amino acids long. The fragment of the immediate early protein may be at least about 250 amino acids long. The fragment of the immediate early protein may be at least about 300 amino acids long. The fragment of the immediate early protein may be at least about 350 amino acids long. The fragment of the immediate early protein may be at least about 400 amino acids long. The fragment of the immediate early protein may be at least about 450 amino acids long. The fragment of the immediate early protein may be at least about 500 amino acids long. The fragment of the immediate early protein may be at least about 550 amino acids long. The fragment of the immediate early protein may be at least about 600 amino acids long. The fragment of the immediate early protein may be at least about 650 amino acids long. The fragment of the immediate early protein may be at least about 700 amino acids long. The fragment of the immediate early protein may be at least about 750 amino acids long. The fragment of the immediate early protein may be at least about 800 amino acids long. The fragment of the immediate early protein may be at least about 850 amino acids long. The fragment of the immediate early protein may be at least about 900 amino acids long. The fragment of the immediate early protein may be at least about 950 amino acids long. The fragment of the immediate early protein may be at least about 1000 amino acids long. The fragment of the immediate early protein may be at least about 1100 amino acids long. The fragment of the immediate early protein may be at least about 1200 amino acids long. The fragment of the immediate early protein may be at least about 1300 amino acids long. The fragment of the immediate early protein may be at least about 1400 amino acids long. The fragment of the immediate early protein may be at least about 1500 amino acids long.

In embodiments, the immediate early Herpes virus protein is ICP0. The ICP0 may be derived from an HHV as described herein (e.g. HHV-1, HHV-2, HHV-3, HHV-4, HHV-5, HHV-6, HHV-7, or HHV-8) and preferably from HHV-1 or HHV-2. In embodiments, the ICP0 corresponds to SEQ ID NO: 4, including antigenic fragments thereof. The ICP0 may include an antigenic site. The ICP0 may include two or more antigenic sites. In embodiments, the ICP0 is a deactivated-ICP0. The deactivated-ICP0 may further include modifications as described herein, including mutation of at least one residue in SEQ ID NO: 4. (e.g. a deactivated-modified ICP0). The mutation may be located in a RING finger domain of the ICP0. In embodiments, the RING finger domain corresponds to residues about 125 to about 165 of SEQ ID NO: 4. In embodiments, the ICP0 is a modified-ICP0. The modified-ICP0 may be chemically modified as described herein, posttranslationally modified as described herein, or truncated (e.g. forming a “truncated-modified ICP0 having deletion of amino acid residues from the N-terminus and/or C-terminus of SEQ ID NO: 4). In embodiments, the modified-ICP0 includes mutation of an amino acid residue. The residue may be located in the RING finger domain as described above. In embodiments, the mutation corresponds to a point mutation of Cys125 or Cys165 of SEQ ID NO: 4. In embodiments, the mutation of a modified-ICP0 may include a deactivating mutation.

In embodiments, the ICP0 is a fragment of a full-length ICP0 which retains antigenicity. The fragment of the ICP0 may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the ICP0 may be at least about 50 amino acids long. The fragment of the ICP0 may be at least about 100 amino acids long. The fragment of the ICP0 may be at least about 150 amino acids long. The fragment of the ICP0 may be at least about 200 amino acids long. The fragment of the ICP0 may be at least about 250 amino acids long. The fragment of the ICP0 may be at least about 300 amino acids long. The fragment of the ICP0 may be at least about 350 amino acids long. The fragment of the ICP0 may be at least about 400 amino acids long. The fragment of the ICP0 may be at least about 450 amino acids long. The fragment of the ICP0 may be at least about 500 amino acids long. The fragment of the ICP0 may be at least about 550 amino acids long. The fragment of the ICP0 may be at least about 600 amino acids long. The fragment of the ICP0 may be at least about 650 amino acids long. The fragment of the ICP0 may be at least about 700 amino acids long. The fragment of the ICP0 may be at least about 750 amino acids long. The fragment of the ICP0 may be at least about 800 amino acids long. The fragment of the ICP0 may be at least about 850 amino acids long.

In embodiments, the immediate early Herpes virus protein is IE1. The IE1 may be derived from an HHV as described herein (e.g. HHV-1, HHV-2, HHV-3, HHV-4, HHV-5, HHV-6, HHV-7, or HHV-8) and preferably from HHV-5. The IE1 may be derived from HCMV. In embodiments, the IE1 corresponds to SEQ ID NO: 6, including antigenic fragments thereof. The IE1 may include an antigenic site. The IE1 may include two or more antigenic sites. In embodiments, the IE1 is a deactivated-IE1. The deactivated-IE1 may further include modifications as described herein, including mutation of an amino acid residue in SEQ ID NO: 6. (e.g. a deactivated-modified IE1). In embodiments, the IE1 is a modified-IE1. The modified-IE1 may be chemically modified as described herein, posttranslationally modified as described herein, or truncated (e.g. forming a “truncated-modified-IE1 having deletion of amino acid residues from the N-terminus and/or C-terminus of SEQ ID NO: 6). In embodiments, the modified-IE1 includes mutation of an amino acid residue. In embodiments, the mutation of a modified-IE1 may include a deactivating mutation.

In embodiments, the IE1 is a fragment of a full-length IE1 which retains antigenicity. The fragment of the IE1 may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the IE1 may be at least about 50 amino acids long. The fragment of the IE1 may be at least about 100 amino acids long. The fragment of the IE1 may be at least about 150 amino acids long. The fragment of the IE1 may be at least about 200 amino acids long. The fragment of the IE1 may be at least about 250 amino acids long. The fragment of the IE1 may be at least about 300 amino acids long. The fragment of the IE1 may be at least about 350 amino acids long. The fragment of the IE1 may be at least about 400 amino acids long. The fragment of the IE1 may be at least about 450 amino acids long. The fragment of the IE1 may be at least about 500 amino acids long.

In embodiments, the immediate early Herpes virus protein is ICP4. The ICP4 may be derived from an HHV as described herein (e.g. HHV-1, HHV-2, HHV-3, HHV-4, HHV-5, HHV-6, HHV-7, or HHV-8) and preferably from HHV-1 or HHV-2. In embodiments, the ICP4 corresponds to SEQ ID NO: 5, including antigenic fragments thereof. The ICP4 may include an one antigenic site. The ICP4 may be a truncated-ICP4 (e.g. a ICP4 having deletion of amino acid residues from the N-terminus and/or C-terminus of SEQ ID NO: 5).

In embodiments, the ICP4 is a fragment of a full-length ICP4 which retains antigenicity. The fragment of the ICP4 may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the ICP4 may be at least about 50 amino acids long. The fragment of the ICP4 may be at least about 100 amino acids long. The fragment of the ICP4 may be at least about 150 amino acids long. The fragment of the ICP4 may be at least about 200 amino acids long. The fragment of the ICP4 may be at least about 250 amino acids long. The fragment of the ICP4 may be at least about 300 amino acids long. The fragment of the ICP4 may be at least about 350 amino acids long. The fragment of the ICP4 may be at least about 400 amino acids long. The fragment of the ICP4 may be at least about 450 amino acids long. The fragment of the ICP4 may be at least about 500 amino acids long. The fragment of the ICP4 may be at least about 550 amino acids long. The fragment of the ICP4 may be at least about 600 amino acids long. The fragment of the ICP4 may be at least about 650 amino acids long. The fragment of the ICP4 may be at least about 700 amino acids long. The fragment of the ICP4 may be at least about 750 amino acids long. The fragment of the ICP4 may be at least about 800 amino acids long. The fragment of the ICP4 may be at least about 850 amino acids long. The fragment of the ICP4 may be at least about 900 amino acids long. The fragment of the ICP4 may be at least about 950 amino acids long. The fragment of the ICP4 may be at least about 1000 amino acids long. The fragment of the ICP4 may be at least about 1100 amino acids long. The fragment of the ICP4 may be at least about 1200 amino acids long. The fragment of the ICP4 may be at least about 1300 amino acids long. The fragment of the ICP4 may be at least about 1400 amino acids long.

In embodiments, about the first 25 amino acids of SEQ ID NO: 5 are deleted. In embodiments, about the first 50 amino acids of SEQ ID NO: 5 are deleted. In embodiments, about the first 75 amino acids of SEQ ID NO: 5 are deleted. In embodiments, about the first 100 amino acids of SEQ ID NO: 5 are deleted. In embodiments, about the first 150 amino acids of SEQ ID NO: 5 are deleted. In embodiments, about the first 125 amino acids of SEQ ID NO: 5 are deleted. In embodiments, about the first 150 amino acids of SEQ ID NO: 5 are deleted. In embodiments, about the first 200 amino acids of SEQ ID NO: 5 are deleted. In embodiments, about the first 250 amino acids of SEQ ID NO: 5 are deleted. In embodiments, about the first 300 amino acids of SEQ ID NO: 5 are deleted. In embodiments, about the first 350 amino acids of SEQ ID NO: 5 are deleted. In embodiments, about the first 400 amino acids of SEQ ID NO: 5 are deleted. In embodiments, about the first 450 amino acids of SEQ ID NO: 5 are deleted. In embodiments, about the first 500 amino acids of SEQ ID NO: 5 are deleted. In embodiments, about the first 550 amino acids of SEQ ID NO: 5 are deleted. In embodiments, about the first 600 amino acids of SEQ ID NO: 5 are deleted. In embodiments, the first 599 residues are deleted.

In embodiments, between about 5 and 100 of the first amino acids are deleted. In embodiments, between about 5 and 200 of the first amino acids are deleted. In embodiments, between about 5 and 300 of the first amino acids are deleted. n embodiments, between about 5 and 400 of the first amino acids are deleted. In embodiments, between about 5 and 500 of the first amino acids are deleted. In embodiments, between about 5 and 600 of the first amino acids are deleted. In embodiments, between about 25 and 100 of the first amino acids are deleted. In embodiments, between about 25 and 200 of the first amino acids are deleted. In embodiments, between about 25 and 300 of the first amino acids are deleted. In embodiments, between about 25 and 400 of the first amino acids are deleted. In embodiments, between about 25 and 500 of the first amino acids are deleted. In embodiments, between about 25 and 600 of the first amino acids are deleted. In embodiments, between about 50 and 100 of the first amino acids are deleted. In embodiments, between about 50 and 200 of the first amino acids are deleted. In embodiments, between about 50 and 300 of the first amino acids are deleted. In embodiments, between about 50 and 400 of the first amino acids are deleted. In embodiments, between about 50 and 500 of the first amino acids are deleted. In embodiments, between about 50 and 600 of the first amino acids are deleted. In embodiments, between about 100 and 200 of the first amino acids are deleted. In embodiments, between about 100 and 300 of the first amino acids are deleted. In embodiments, between about 100 and 400 of the first amino acids are deleted. In embodiments, between about 100 and 500 of the first amino acids are deleted. In embodiments, between about 100 and 600 of the first amino acids are deleted. In embodiments, the truncated-ICP4 corresponds to residues 600-1318 of SEQ ID NO: 5 (i.e. residues corresponding to residues 1-599 are deleted).

The ICP4 may include two or more antigenic sites. In embodiments, the ICP4 is a deactivated-ICP4. The deactivated-ICP4 may further include modifications as described herein, including mutation of an amino acid residue in SEQ ID NO: 5 (e.g. a deactivated-modified ICP4). In embodiments, the ICP4 is a modified-ICP4. The modified-ICP4 may be chemically modified as described herein, posttranslationally modified as described herein, or truncated (e.g. forming a “truncated-modified ICP4 having deletion of amino acid residues from the N-terminus and/or C-terminus of SEQ ID NO: 5 and mutation of an amino acid residue within the remaining truncated-modified ICP4 (i.e. the mutation is not within the deleted amino acid residues). In embodiments, the truncated-modified ICP4 includes residues corresponding to residues 600-1318 of SEQ ID NO: 5 (i.e. residues corresponding to residues 1-599 are deleted). The truncated-modified ICP4 may further include mutation of the sequence corresponding to residue positions 1063-1070 of SEQ ID NO: 5 (e.g. “ADWPADGP”). In embodiments, the modified ICP4 includes a mutation. The mutation may be within the sequence corresponding to residue positions 1063-1070 of SEQ ID NO: 5. The mutation may be a deactivating mutation.

In embodiments, the sequence corresponding to residue positions 1063-1070 of SEQ ID NO: 5 includes a conservative mutation as described herein. In embodiments, the sequence corresponding to residue positions 1063-1070 of SEQ ID NO: 5 may be formula (I):

H₂N-Aaa1-Aaa2-Aaa3-Pro-Aaa4-Aaa5-Aaa6-Pro-COOH (I).

Aaa1 is Gly, Cys, Ser, or Val. Aaa2 is Ala, Gly, Leu, Ile, Ser, or Val. Aaa3 is Tip, Phe or Tyr. Aaa4 is Ala, Gly, or Val. Aaa5 is Ala, Gly, Leu, Ile, Ser, or Val. Aaa6 is Ala, Gly, or Val. In embodiments formula (I) is N₂N-CAWPAAAP-COOH.

In embodiments, the immediate early Herpes virus protein is IE2. The IE2 may be derived from an HHV as described herein (e.g. HHV-1, HHV-2, HHV-3, HHV-4, HHV-5, HHV-6, HHV-7, or HHV-8) and preferably from HHV-5. In embodiments, the IE2 corresponds to SEQ ID NO: 7, including antigenic fragments thereof. The IE2 may include an antigenic site. The IE2 may be a truncated-IE2 (e.g. a IE2 having deletion of amino acid residues from the N-terminus and/or C-terminus of SEQ ID NO: 7). The IE2 may include two or more antigenic sites. In embodiments, the IE2 is a deactivated-IE2. The deactivated-IE2 may further include modifications as described herein, including mutation of an amino acid residue in SEQ ID NO: 7 (e.g. a deactivated-modified IE2). In embodiments, the IE2 is a modified-IE2. The modified-IE2 may be chemically modified as described herein, posttranslationally modified as described herein, or truncated (e.g. forming a “truncated-modified IE2 having deletion of amino acid residues from the N-terminus and/or C-terminus of SEQ ID NO: 7 and mutation of an amino acid residue within the remaining truncated-modified IE2 (i.e. the mutation is not within the deleted amino acid residues). The mutation may be a deactivating mutation.

In embodiments, the IE2 is a fragment of a full-length IE2 which retains antigenicity. The fragment of the IE2 may be at least about 10, 20, 50, 100, 150, 200, 250, 300, 350, 400, 450, 500, 550, 600, 650, 700, 750, 800, 850, 900, 1000, 1100, 1200, 1300, 1400, or 1500 amino acids. The fragment of the IE2 may be at least about 50 amino acids long. The fragment of the IE2 may be at least about 100 amino acids long. The fragment of the IE2 may be at least about 150 amino acids long. The fragment of the IE2 may be at least about 200 amino acids long. The fragment of the IE2 may be at least about 250 amino acids long. The fragment of the IE2 may be at least about 300 amino acids long. The fragment of the IE2 may be at least about 350 amino acids long. The fragment of the IE2 may be at least about 400 amino acids long. The fragment of the IE2 may be at least about 450 amino acids long. The fragment of the IE2 may be at least about 500 amino acids long. The fragment of the IE2 may be at least about 550 amino acids long. The fragment of the IE2 may be at least about 600 amino acids long.

### II. Pharmaceutical Compositions

In embodiments, the vaccine composition is formulated as a pharmaceutical composition. The pharmaceutical composition includes an inactivated Herpes virus as described herein including embodiments thereof, a recombinant-Herpes virus-protein as described herein including embodiments thereof, a vaccine adjuvant as described herein including embodiments thereof, and a pharmaceutically acceptable excipient.

The pharmaceutical composition may include an inactivated Herpes virus as described herein, including embodiments thereof, a recombinant-Herpes virus-protein as described herein including embodiments thereof, a LPS-adjuvant or an aluminum-based mineral salt adjuvant, and a pharmaceutically acceptable excipient. The pharmaceutical composition may include an inactivated Herpes virus as described herein, including embodiments thereof, a recombinant-Herpes virus-protein as described herein including embodiments thereof, a LPS-adjuvant and an aluminum-based mineral salt adjuvant, and a pharmaceutically acceptable excipient. The pharmaceutical composition may include an inactivated Herpes virus as described herein, including embodiments thereof, a recombinant-Herpes virus-protein as described herein including embodiments thereof, a MPL and an aluminum-based mineral salt adjuvant (e.g. aluminum hydroxide or aluminum phosphate), and a pharmaceutically acceptable excipient.

The pharmaceutical compositions described herein, including embodiments thereof can be administered alone or can be coadministered to the patient. Coadministration is meant to include simultaneous or sequential administration of the compositions individually or in combination (more than one composition). Thus, the preparations can also be combined, when desired, with other active substances (e.g. to reduce metabolic degradation, increase immune response (e.g. adjuvants)). An example of co-administration of the vaccine compositions described herein is a prime-boost method of administration.

The pharmaceutical compositions described herein can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The pharmaceutical compositions described herein thereof can also be administered by injection, that is, intravenously, intramuscularly, intracutaneously, subcutaneously, intraduodenally, or intraperitoneally. Also, the pharmaceutical compositions described herein can be administered by inhalation, for example, intranasally. Additionally, the pharmaceutical compositions described herein can be administered transdermally. It is also envisioned that multiple routes of administration (e.g., intramuscular, oral, transdermal, mucosal, intranasal, intrarectal, intravaginal, subcutaneous, transcutaneous, topical, intradermal) can be used to administer the compositions of the invention.

For preparing the pharmaceutical compositions described herein from the vaccine compositions described herein, including embodiments thereof, pharmaceutically acceptable carriers may be added which can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid in a mixture with the finely divided active component (e.g. a vaccine composition). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active composition (i.e., dosage). Pharmaceutical preparations of the vaccine compositions described herein can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active composition is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The vaccine compositions described herein, including embodiments thereof can also be incorporated into liposomes or administered via transdermal pumps or patches. Pharmaceutical admixtures suitable for use herein are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component (e.g. vaccine compositions described herein, including embodiments, examples) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations described herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical compositions described herein are preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Some compositions may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

Pharmaceutical compositions described herein include compositions wherein the active ingredient (e.g. vaccine compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent Herpes virus (e.g. HSV, HHV-1, HHV-2, HHV-3, HHV-4, HHV-5, HHV-6, HHV-7, or HHV-8) infection, prevent HSV (e.g. HSV1 and/or HSV2) infection, prevent HCMV infection and/or reducing, eliminating, or slowing the progression of disease symptoms (e.g. Herpes virus (e.g. HSV, HHV-1, HHV-2, HHV-3, HHV-4, HHV-5, HHV-6, HHV-7, or HHV-8) infection, HSV (e.g. HSV1 and/or HSV2) infection, or HCMV infection). Determination of a therapeutically or prophylactically effective amount of a composition of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. HV (e.g. HSV, HHV-1, HHV-2, HHV-3, HHV-4, HHV-5, HHV-6, HHV-7, or HHV-8) infection, HSV (e.g. HSV1 and/or HSV2) infection, or HCMV infection), kind of concurrent treatment, complications from the disease being treated or other health-related problems. Other therapeutic regimens or agents can be used in conjunction with the methods and compositions of Applicants' invention. Adjustment and manipulation of established dosages (e.g., frequency and duration) are well within the ability of those skilled in the art.

For vaccine compositions described herein, the therapeutically effective amount can be initially determined from cell culture assays. Target concentrations will be those concentrations of active composition(s) that are capable of achieving the methods described herein, as measured using the methods described herein or known in the art.

As is well known in the art, therapeutically or prophylactically effective amounts for use in humans can also be determined from animal models. For example, a dose for humans can be formulated to achieve a concentration that has been found to be effective in animals. The dosage in humans can be adjusted by monitoring compositions effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the composition being employed. The dose administered to a patient, in the context of the present invention should be sufficient to effect a beneficial therapeutic or prophylactic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered composition effective for the particular clinical indication being treated or prevented. This will provide a therapeutic or prophylactic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active composition by considering factors such as composition potency, relative bioavailability, patient body weight, presence and severity of adverse side effects, preferred mode of administration and the toxicity profile of the selected agent. This planning may include a prime boost vaccine administration as described herein.

The ratio between toxicity and therapeutic effect for a particular composition is its therapeutic index and can be expressed as the ratio between LD₅₀ (the amount of composition lethal in 50% of the population) and ED₅₀ (the amount of composition effective in 50% of the population). Compositions that exhibit high therapeutic indices are preferred. Therapeutic index data obtained from cell culture assays and/or animal studies can be used in formulating a range of dosages for use in humans. The dosage of such compositions preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p.1, 1975. The exact formulation, route of administration and dosage can be chosen by the individual physician in view of the patient's condition and the particular method in which the composition is used.

### III. Methods

Also provided herein are methods of using the vaccine compositions described herein. In one aspect, the method includes treating a Herpes disease in a subject in need thereof by administering a therapeutically effective amount of a vaccine composition as described herein, including embodiments thereof. The Herpes disease may be a Herpes virus as described herein. Thus, in embodiments, the Herpes disease is an HHV-1 infection, HHV-2 infection, HHV-3 infection, HHV-4 infection, HHV-5 infection, HHV-6 infection, HHV-7 infection or HHV-8 infection. The Herpes disease may be an HHV-1 infection or HHV-2 infection. The Herpes disease may be an HHV-1 infection. The Herpes disease may be an HHV-2 infection. The Herpes disease may be a HSV infection (e.g. a HSV-1 infection or a HSV-2 infection). The Herpes disease may be a HSV-1 infection. The Herpes disease may be a HSV-2 infection. The Herpes disease may be a HSV-1 infection and a HSV-2 infection. The Herpes disease may be an HHV-5 infection. The Herpes disease may be a HCMV infection.

Provided herein are methods of preventing a Herpes disease in a subject in need of prevention. In one aspect, the method includes administering a prophylactically effective amount of a vaccine composition as described herein, including embodiments thereof. Thus, in embodiments, the methods herein include treating and/or preventing a Herpes disease or infection by a Herpes virus.

In embodiments of the methods described herein, the Herpes disease is herpetic gingivostomatitis, herpes labialis, herpes genitalis, herpetic whitlow, herpes gladiatorum, herpesviral encephalitis, herpesviral meningitis, herpes esophagitis, herpes keratitis, Bell's palsy, Mollaret's meningitis, herpes rugbeiorum, eczema herpeticum, herpetic neuralgia, post-herpetic neuralgia, or disseminated neonatal herpes. The Herpes disease may be herpetic gingivostomatitis. The Herpes disease may be herpes labialis. The Herpes disease may be herpes genitalis. The Herpes disease may be herpetic whitlow. The Herpes disease may be herpes gladiatorum. The Herpes disease may be herpesviral encephalitis. The Herpes disease may be herpesviral meningitis. The Herpes disease may be herpes esophagitis. The Herpes disease may be herpes keratitis. The Herpes disease may be Bell's palsy. The Herpes disease may be Mollaret's meningitis. The Herpes disease may be herpes rugbeiorum. The Herpes disease may be eczema herpeticum. The Herpes disease may be herpetic neuralgia. The Herpes disease may be post-herpetic neuralgia. The Herpes disease may be disseminated neonatal herpes.

The vaccine composition is as described herein, including embodiments thereof. In embodiments of the methods described herein, the inactivated Herpes virus of the vaccine composition is HHV-1, HHV-2, HSV-1, HSV-2, HHV-5, or HCMV. In embodiments of the methods described herein, the vaccine adjuvant is as described herein, including embodiments thereof. In embodiments of the methods described herein, the vaccine adjuvant is MPL and an aluminum-based mineral salt adjuvant (e.g. aluminum hydroxide or aluminum phosphate). The recombinant-Herpes virus protein is as described herein, including embodiments thereof. In embodiments of the methods described herein, the recombinant-Herpes virus protein is an envelope protein, a capsid protein, a matrix protein, or an immediate early Herpes virus protein, including embodiments thereof. In embodiments of the methods described herein, the recombinant-Herpes virus protein is an envelope protein or an immediate early Herpes virus protein as described herein, including embodiments thereof. In embodiments of the methods described herein, the recombinant-Herpes virus protein is an envelope protein. In embodiments of the methods described herein, the recombinant-Herpes virus protein is an immediate early Herpes virus protein. In embodiments of the methods described herein, the envelope protein is as described herein, including embodiments thereof, and is preferably gD (e.g. gD2), gB, gG, gH, or gL. In embodiments of the methods described herein, the immediate early Herpes virus protein is as described herein, including embodiments thereof, and is preferably ICP0, ICP4, IE1, or IE2. In embodiments of the methods described herein, the recombinant-Herpes virus protein generates neutralizing antibodies (e.g. an antibody that inhibits the infectivity of a virus (e.g. HV virus as described herein, including embodiments thereof).

In embodiments of the methods described herein, the vaccine composition is compared against a control composition to determine its efficacy. In embodiments of the methods described herein, administration of the vaccine compositions described herein reduces, prevents, or eliminates recurrence of the Herpes disease (e.g. HSV-1, HSV-2, or HCMV). In embodiments of the methods described herein, administration of the vaccine composition reduces viral shedding. Administration of the vaccine compositions described herein may eliminate viral shedding. Administration of the vaccine compositions described herein may reduce the duration or amount of viral shedding. Administration of the vaccine compositions described herein may prevent viral shedding. In embodiments of the methods described herein, administration of the vaccine compositions described herein reduces the frequency of lesions. The administration of the vaccine compositions described herein may reduce the duration of lesion occurrence. The administration of the vaccine compositions described herein may prevent lesions.

In embodiments of the methods described herein, administration of the vaccine compositions described herein reduces viral shedding at least about 2-log fold compared to the control composition. Administration of the vaccine compositions described herein may reduce viral shedding at least about 3-log fold compared to the control composition. Administration of the vaccine compositions described herein may reduce viral shedding at least about 4-log fold compared to the control composition. Administration of the vaccine compositions described herein may reduce viral shedding at least about 5-log fold compared to the control composition. Administration of the vaccine compositions described herein may reduce viral shedding at least about 6-log fold compared to the control composition. Administration of the vaccine compositions described herein may reduce viral shedding at least about 7-log fold compared to the control composition. Administration of the vaccine compositions described herein may reduce viral shedding at least about 8-log fold compared to the control composition. Administration of the vaccine compositions described herein may reduce viral shedding at least about 9-log fold compared to the control composition. Administration of the vaccine compositions described herein may reduce viral shedding at least about 10-log fold compared to the control composition. In embodiments of the methods described herein, the viral shedding is determined through vaginal virus titers.

In embodiments of the methods described herein, a subject is tested for the presence of HV-specific antibodies (e.g. an antibody produced by the subject in response to vaccination to HSV-1, HSV-2, HCMV). The testing may be performed using techniques known in the art such as ELISA-based testing. In embodiments of the methods described herein, administration of the vaccine to the subject results in expression of cytokines. The cytokines may be Th1 or Th2 cytokines (e.g. cytokines from Th1 or Th2 cells).

In embodiments of the methods described herein, the vaccine composition is formulated for intramuscular administration. The vaccine composition may be formulated for intradermal administration. The vaccine composition may be formulated for mucosal administration. The vaccine composition may be formulated for intranasal administration. The vaccine composition may be formulated for intrarectal administration. The vaccine composition may be formulated for intravaginal administration. The vaccine composition may be formulated for topical administration. The vaccine composition may be formulated for subcutaneous administration. The vaccine composition may be formulated for transcutaneous administration.

In embodiments of the methods described herein, the administered vaccine compositions described herein vaccinate a recipient of the vaccine against HV infection for up to 1, 2, 3, 4, 5, 6, 7, 8, 9, 10, 11, 12, 13, 14, 15, 16, 17, 18, 19, 20, or more years. In embodiments the administered vaccine compositions described herein protect a patient against one or more strains of the HV. In embodiments of the methods described herein, the administered vaccine compositions described herein treat a patient for one or more strains of the HV. In embodiments of the methods described herein, the administered vaccine compositions described herein prevent a patient from being infected by, contracting, getting, or having one or more strains of the HV. In embodiments of the methods described herein, the administered vaccine compositions described herein prevent a patient administered the vaccine from being infected by, contracting, getting, or having more than one strain of the HV.

In embodiments of the methods described herein, the vaccine compositions of the methods herein are administered as a pharmaceutical composition optionally comprising one or more pharmaceutically acceptable excipients as described herein. The vaccine compositions of the methods may be supplied as sterile solutions or as powders which require solubilizing before administration.

In embodiments of the methods described herein, the subject is a HSV-1 or HSV-2 seropositive subject (i.e. a sample from a subject tests positive for a Herpes virus such as HSV or HCMV). In embodiments, the subject is HSV-1 seropositive. In embodiments, the subject is HSV-2 seropositive. In embodiments, the subject is HSV-1 and HSV-2 seropositive. In embodiments, the subject is HCMV seropositive.

In embodiments, the methods herein further include administering a prime boost vaccine as described herein. In embodiments, the prime boost vaccine is administered about 1, 2, 3, 4, 5, 6, 7, 10, 15, 20, 25, 30, 35, 40, 45, 50, 55, 60, 65, 70, 75, 80, 85, or 90 days within the administration of the vaccine compositions described herein. In embodiments, the prime boost vaccine is administered about 1, 2, 3, 4, 5, 6, 7, 8, 9, 10, 11, or 12 weeks within the administration of the vaccine compositions described herein. In embodiments, the prime boost vaccine is administered about 1, 2, 3, 4, 5, 6, 7, 8, 9, 10, 11, 12 months within the administration of the vaccine compositions described herein.

The vaccine compositions described herein may be administered as described above, with a prime boost vaccine. Exemplary prime boost vaccines include those described in U.S. Pat. No. 8,501,194, which is herein incorporated in full by reference. In embodiments, the prime boost vaccine is a DNA-vaccine (e.g. a vaccine having an antigenic component that is a DNA sequence). The DNA-vaccine may include a polynucleotide component (e.g. a gene or a fragment thereof) of an endogenous-Herpes virus protein. The prime boost vaccine may include one or more gene portions of one or more of endogenous-Herpes virus proteins. Exemplary genes of endogenous-Herpes virus proteins that may be used in prime boost vaccines in the methods described herein, include but are not limited to, UL1, UL2, UL3, UL4, UL5, UL6, UL7, UL8, UL9, UL10, UL11, UL12, UL13, UL14, UL15, UL16, UL17, UL18, UL19, UL20, UL21, UL22, UL23, UL24, UL25, UL26, UL27, UL28, UL29, UL30, UL31, UL32, UL33, UL34, UL35, UL36, UL37, UL38, UL39, UL40, UL41, UL42, UL43, UL44, UL45, UL46, UL47, UL48, UL49, UL50, UL51, UL52, UL53, UL54, UL55, UL56, US1, US2, US3, US4, US5, US6, US7, US8, US9, US10, US11, US12, RS1, ICP0, LRP1, LRP2, RL1, or LAT. In embodiments, the DNA-vaccine includes one or more endogenous-Herpes virus proteins, or fragments thereof, of the aforementioned genes. The DNA component or the polypeptide component may be antigenic. In embodiments, the DNA component and the polypeptide component may be antigenic. In embodiments, the prime boost vaccine includes a recombinant-Herpes virus protein as described herein. In embodiments, the recombinant-Herpes virus protein is gD2t as described herein. In embodiments, the prime includes DNA encoding non-structural proteins as described herein.

Also provided herein are kits which include a vaccine composition as described herein, including embodiments thereof and an administration device. The kits may include containers, buffers, and solutions (which may optionally be sterilized) useful for the administration of the vaccine compositions according to the methods described herein. The inactivated Herpes virus is as described herein, including embodiments thereof. The recombinant-Herpes virus protein is as described herein, including embodiments thereof. The vaccine adjuvant is as described herein, including embodiments thereof. In embodiments of the kit, vaccine composition is administered in a prime-boost administration as described herein, including embodiments thereof.

### IV. Embodiments

### Embodiment P1

A Herpes virus vaccine comprising an inactivated Herpes virus and a recombinant Herpes virus protein.

### Embodiment P2

The vaccine of embodiment P1, further comprising a lipopolysaccharide (LPS)-derived adjuvant and an aluminum-based mineral salt adjuvant.

### Embodiment P3

The vaccine of embodiment P2, wherein said LPS-derived adjuvant is MPL and said aluminum-based mineral salt adjuvant is aluminum hydroxide adjuvant or aluminum phosphate adjuvant.

### Embodiment P4

The vaccine of embodiment P1, wherein said recombinant Herpes virus protein forms part of said inactivated Herpes virus, thereby forming an incorporated Herpes virus protein.

### Embodiment P5

The vaccine of embodiment P4, wherein said recombinant Herpes virus protein is present at an amount at least 10-fold the amount of said incorporated Herpes virus protein.

### Embodiment P6

The vaccine of embodiment P1, wherein said recombinant Herpes virus protein is an envelope protein.

### Embodiment P7

The vaccine of embodiment P1, wherein said envelope binding protein binds a neutralizing antibody.

### Embodiment P8

The vaccine of embodiment P6, wherein said envelope protein is a glycoprotein.

### Embodiment P9

The vaccine of embodiment P8, wherein said glycoprotein is glycoprotein D.

### Embodiment P10

The vaccine of embodiment P1, wherein said recombinant Herpes virus protein is an immediate early Herpes virus protein.

### Embodiment P11

The vaccine of embodiment P10, wherein said immediate early Herpes virus protein is an ubiquitin ligase.

### Embodiment P12

The vaccine of embodiment P11, wherein said ubiquitin ligase is ICP0.

### Embodiment P13

The vaccine of embodiment P10, wherein said immediate early Herpes virus protein is a transcriptional activator.

### Embodiment P14

The vaccine of embodiment P13, wherein said transcriptional activator is ICP4.

### Embodiment P15

A method of treating or preventing a disease in a patient in need of such treatment or prevention, said method comprising administering a therapeutically or prophylactically effective amount of the Herpes virus vaccine of one of embodiments P1-P14.

### Embodiment P16

The method of embodiment P15, wherein said disease is selected from the group consisting of herpetic gingivostomatitis, herpes labialis, herpes genitalis, herpetic whitlow, herpes gladiatorum, herpesviral encephalitis, herpesviral meningitis, herpes esophagitis, herpes keratitis, Bell's palsy, Mollaret's meningitis, herpes rugbeiorum, eczema herpeticum, herpetic neuralgia, and post-herpetic neuralgia.

### Embodiment P17

A kit comprising the Herpes virus vaccine of one of embodiments P1-P14 and instructions for administering said Herpes virus vaccine.

### Embodiment P18

The kit of embodiment P17, wherein said Herpes virus vaccine is administered in a prime boost administration.

### Embodiment 1

A vaccine composition comprising an inactivated Herpes virus, a recombinant Herpes virus-protein, and a vaccine adjuvant.

### Embodiment 2

The vaccine composition of embodiment 1, wherein said vaccine adjuvant is a lipopolysaccharide (LPS)-adjuvant.

### Embodiment 3

The vaccine composition of any one of embodiments 1 to 2, further comprising an aluminum-based mineral salt adjuvant.

### Embodiment 4

The vaccine composition of any one of embodiments 1 to 3, wherein said LPS-adjuvant is monophosphoryl lipid A (MPL) and said aluminum based mineral salt adjuvant is aluminum hydroxide or aluminum phosphate.

### Embodiment 5

The vaccine composition of any one of embodiments 1 to 4, wherein said vaccine adjuvant is an aluminum-based mineral salt adjuvant.

### Embodiment 6

The vaccine composition of any one of embodiments 1 to 5, wherein said recombinant Herpes virus protein is present at an amount at least 10-fold greater than a corresponding endogenous Herpes virus protein.

### Embodiment 7

The vaccine composition of any one of embodiments 1 to 6, wherein said recombinant Herpes virus protein is present in an amount from about 10-fold greater to about 50-fold greater than the corresponding endogenous Herpes virus protein.

### Embodiment 8

The vaccine composition of any one of embodiments 1 to 7, wherein said recombinant Herpes virus protein comprises an antigenic site.

### Embodiment 9

The vaccine composition of any one of embodiments 1 to 8, wherein said recombinant Herpes virus protein is a deactivated-recombinant Herpes virus protein.

### Embodiment 10

The vaccine composition of any one of embodiments 1 to 9, wherein said recombinant Herpes virus protein is an envelope protein.

### Embodiment 11

The vaccine composition of any one of embodiments 1 to 10, wherein said envelope protein is a glycoprotein.

### Embodiment 12

The vaccine composition of any one of embodiments 1 to 11, wherein said glycoprotein is a glycoprotein B, a glycoprotein D, a glycoprotein G, a glycoprotein H, or a glycoprotein L.

### Embodiment 13

The vaccine composition of any one of embodiments 1 to 11, wherein said glycoprotein is a glycoprotein D or a glycoprotein B.

### Embodiment 14

The vaccine composition of any one of embodiments 1 to 11, wherein said glycoprotein is a glycoprotein D2.

### Embodiment 15

The vaccine composition of any one of embodiments 1 to 14, wherein said glycoprotein D2 comprises an antigenic site.

### Embodiment 16

The vaccine composition of any one of embodiments 1 to 15, wherein said glycoprotein D2 is a truncated-glycoprotein D2.

### Embodiment 17

The vaccine composition of any one of embodiments 1 to 16, wherein said recombinant Herpes virus protein is an immediate early Herpes virus protein.

### Embodiment 18

The vaccine composition of any one of embodiments 1 to 17, wherein said immediate early Herpes virus protein is ICP0.

### Embodiment 19

The vaccine composition of any one of embodiments 1 to 18, wherein said ICP0 comprises at least one antigenic site.

### Embodiment 20

The vaccine composition of any one of embodiments 1 to 19, wherein said ICP0 is a deactivated-ICP0.

### Embodiment 21

The vaccine composition of any one of embodiments 1 to 20, wherein said ICP0 is a modified-ICP0.

### Embodiment 22

The vaccine composition of any one of embodiments 1 to 21, wherein said modified ICP0 is a deactivated-modified ICP0.

### Embodiment 23

The vaccine composition of any one of embodiments 1 to 21, wherein said modified ICP0 comprises a mutation.

### Embodiment 24

The vaccine composition of any one of embodiments 1 to 23, wherein said mutation is within a RING finger domain of said modified-ICP0.

### Embodiment 25

The vaccine composition of any one of embodiments 1 to 23, wherein said mutation is a point mutation of a residue corresponding to Cys125 or Cys165 of SEQ ID NO: 4.

### Embodiment 26

The vaccine composition of any one of embodiments 1 to 25, wherein said mutation is a deactivating-mutation.

### Embodiment 27

The vaccine composition of embodiment 17, wherein said immediate early Herpes virus protein is IE1.

### Embodiment 28

The vaccine composition of any one of embodiments 17 or 27, wherein said immediate early Herpes virus protein is ICP4.

### Embodiment 29

The vaccine composition of any one of embodiments 27 to 28, wherein said ICP4 comprises an antigenic site.

### Embodiment 30

The vaccine composition of any one of embodiments 27 to 29, wherein said ICP4 is a deactivated-ICP4.

### Embodiment 31

The vaccine composition of any one of embodiments 27 to 30, wherein said ICP4 is a modified-ICP4.

### Embodiment 32

The vaccine composition of any one of embodiments 27 to 31, wherein said modified ICP4 is deactivated-modified ICP4.

### Embodiment 33

The vaccine composition of any one of embodiments 27 to 32, wherein said modified-ICP4 is a truncated-ICP4.

### Embodiment 34

The vaccine composition of any one of embodiments 27 to 33, wherein said modified-ICP4 comprises a mutation of a residue within a sequence corresponding to residue positions 1063-1070 of SEQ ID NO: 5.

### Embodiment 35

The vaccine composition of any one of embodiments 27 to 34, wherein the sequence corresponding to residue positions 1063-1070 of SEQ ID NO: 5 of said modified-ICP4 is CAWPAAAP.

### Embodiment 36

The vaccine composition of any one of embodiments 27 to 34, wherein said mutation is a deactivating-mutation.

### Embodiment 37

The vaccine composition of embodiment 17, wherein said immediate early Herpes virus protein is IE2.

### Embodiment 38

The vaccine composition of any one of embodiments 1 to 37, comprising a single inactivated Herpes virus, wherein said inactivated Herpes virus is HSV-1 or HSV-2.

### Embodiment 39

The vaccine composition of any one of embodiments 1 to 37, wherein said vaccine composition comprises two or more inactivated Herpes viruses, wherein said two or more inactivated Herpes viruses are HSV-1 and HSV-2.

### Embodiment 40

The vaccine composition of any one of embodiments 1 to 37, wherein a single inactivated Herpes virus, wherein said inactivated Herpes virus is HHV-5.

### Embodiment 41

The vaccine composition of any one of embodiments 1 to 40, wherein said vaccine composition is formulated for intramuscular or subcutaneous administration.

### Embodiment 42

The vaccine composition of any one of embodiments 1 to 41, wherein said vaccine composition is formulated as a pharmaceutical composition comprising a pharmaceutically acceptable excipient.

### Embodiment 43

A method of treating a Herpes disease in a subject in need thereof, said method comprising administering a therapeutically effective amount of a vaccine composition according to any one of embodiments 1 to 44.

### Embodiment 44

The method of embodiment 43, wherein said Herpes disease is an HHV-1 infection, HHV-2 infection, HHV-3 infection, HHV-4 infection, HHV-5 infection, HHV-6 infection, HHV-7 infection or HHV-8 infection.

### Embodiment 45

The method of any one of embodiments 43 to 44, wherein said Herpes disease is an HHV-1 infection or HHV-2 infection.

### Embodiment 46

The method of any one of embodiments 43 to 45, wherein said Herpes disease is herpetic gingivostomatitis, herpes labialis, herpes genitalis, herpetic whitlow, herpes gladiatorum, herpesviral encephalitis, herpesviral meningitis, herpes esophagitis, herpes keratitis, Bell's palsy, Mollaret's meningitis, herpes rugbeiorum, eczema herpeticum, herpetic neuralgia, post-herpetic neuralgia, or disseminated neonatal herpes.

### Embodiment 47

The method of any one of embodiments 43 to 46, wherein said Herpes disease is an HHV-5 infection.

### Embodiment 48

The method of any one of embodiments 43 to 47, wherein said method further comprises administering to said subject a prime boost vaccine.

### Embodiment 49

The method of any one of embodiments 43 to 48, wherein said administration is intramuscular administration.

### Embodiment 50

The method of any one of embodiments 43 to 48, wherein said administration is subcutaneous administration.

### Embodiment 51

A method of preventing a Herpes disease in a subject in need thereof, said method comprising administering a prophylactically effective amount of a vaccine composition according to any one of embodiments 1 to 42.

### Embodiment 52

The method of embodiment 51, wherein said Herpes disease is an HHV-1 infection, HHV-2 infection, HHV-3 infection, HHV-4 infection, HHV-5 infection, HHV-6 infection, HHV-7 infection or HHV-8 infection.

### Embodiment 53

The method of any one of embodiments 51 to 52, wherein said Herpes disease is an HHV-1 infection or HHV-2 infection.

### Embodiment 54

The method of any one of embodiments 51 to 53, wherein said Herpes disease is herpetic gingivostomatitis, herpes labialis, herpes genitalis, herpetic whitlow, herpes gladiatorum, herpesviral encephalitis, herpesviral meningitis, herpes esophagitis, herpes keratitis, Bell's palsy, Mollaret's meningitis, herpes rugbeiorum, eczema herpeticum, herpetic neuralgia, post-herpetic neuralgia, or disseminated neonatal herpes.

### Embodiment 55

The method of any one of embodiments 51 to 54, wherein said Herpes disease is an HHV-5 infection.

### Embodiment 56

The method of any one of embodiments 51 to 55, wherein said method further comprises administering to said subject a prime boost vaccine.

### Embodiment 57

The method of any one of embodiments 51 to 56, wherein said administration is intramuscular administration.

### Embodiment 58

The method of any one of embodiments 51 to 56, wherein said administration is subcutaneous administration.

### Embodiment 59

The method of any one of embodiments 51 to 58, wherein said vaccine composition is administered as a prime boost administration.

### Embodiment 60

A kit comprising the vaccine composition of any one of embodiments 1 to 42 and an administration device.

### V. Examples

Several subunit vaccines have been clinically tested for efficacy, and significant protection was not achieved until HSV glycoprotein D2 (gD2), which can elicit neutralizing antibodies, was formulated with a Th1 promoting adjuvant containing monophosphoryl lipid A (MPL) and alum (MPL/Alum). In the first large trial involving HSV-2 discordant couples, this vaccine was protective against disease only in HSV-1 and -2 seronegative women. However, in a follow-up phase III study, this vaccine showed limited protection against genital disease caused by HSV-1 and no protection against HSV-2 disease.

The limited efficacy of these vaccines suggests that neutralizing antibodies may be necessary, but alone, appear insufficient for a vaccine development strategy (2). Thus, without being bound by any particular theory, T cell responses is likely a necessary component to efficacious vaccine development. In the mouse intravaginal HSV-2 challenge model, CD4⁺ T cells and IFN-γ are important mucosal effectors (19) and CD8⁺ T cells are associated with control of ganglionic infection (14). Additionally, CD4⁺ T cell loss correlates with HSV-2 shedding in HIV-1-infected individuals, and HSV-2-specific CD4⁺ and CD8⁺ T cells localize to genital herpes lesions and can clear infected skin keratinocytes (15, 30). Recent transcriptional analysis of CD8+ T cells residing in biopsies of resolved genital lesions at the dermal-epidermal junction proximal to sensory neuron endings demonstrated highly-expressed genes for activation, proliferation, antiviral activity, and metabolism, suggesting an active role of these resident T cells for HSV-2 immunosurveillance (23). Together, these data suggest that an effective HSV-2 vaccine likely relies on robust induction of both HSV-2-specific antibody and T cell responses.

The unique platform discovered herein works for vaccines that can be used for the treatment or prevention of herpesvirus infections. Principles used for making vaccines against acute virus infections (i.e. influenza) cannot be used for designing vaccines against the herpesviruses, which cause persistent infections. In vaccine development for acute viruses, a significant inclusion criterion has been which given viral protein can generate a strong antibody or T-cell response during natural infection. In contrast, immunity following natural infection of persistent viruses may suppress dissemination of the virus and disease, but the virus persists. The herpesviruses are masters at immune evasion and can prevent the host from mounting a fully protective response. During persistent viral infections, the most visible populations of immune cells in infected individuals may be immunodominant, but not immunoprotective. Thus vaccination must be more effective in generating protection than natural infection. In this regard, the identified targets of the CD4 and CD8 T cells that are dominant specifically in HSV seropositive/asymptomatic individuals and HSV exposed/seronegative subjects are likely important.

While past studies into the specificities of T cells against HSV have identified several viral antigens that are elicited in seropositive individuals, more recent studies have identified T cell specificities that predominate in either asymptomatic individuals or HSV exposed individuals who remained seronegative, do not shed virus, but have HSV specific T cells (reviewed in (16)). Notably, among the T cell specificities identified are those against the immediate early (IE) HSV proteins ICP0 (ubiquitin ligase) and ICP4 (transcriptional activator), with CD4+ and CD8+ T cells that react to epitopes found from these IE antigens of both HSV-2 and HSV-1 (16). In addition, HCMV homolog proteins such as IE1 and IE2 likely react in a similar manner. Thus, eliciting immunogenic response using immediate early proteins results in eliciting 1) protective T cells against ICP0 and ICP4 (and IE1 and IE2) as well as 2) protective T and B cell responses to gD2 (or gB), and 3) a broad range of T and B cell responses against an inactivated HSV-2 particle (or HCMV particle) in adjuvant vaccine that has been demonstrated to be highly protective in mouse and guinea pig models. Significantly enhanced control of virus after either prophylactic or therapeutic vaccination can be achieved by providing virus specific immunity both at the mucosal site of entry as well as at the ganglia/mucosal interface.

Applicants discovered, inter alia, that the combined use of novel T cell antigens, such as gD2, gB, ICP0, ICP4, IE1 and IE2, with a highly protective viral particle boost in an adjuvant contributes to the first significant protection against HSV-1 and -2 in clinical trials. ICP0 and ICP4 have been strongly implicated to be key T cell targets in individuals that are protected against either symptomatic GH or in HSV exposed but seronegative individuals. In addition, it has recently been reported that ICP4 can contribute to a protective, therapeutic HSV-2 vaccine in guinea pigs (25). These IE proteins also have a potential advantage as target antigens due to their immediate expression after viral infection allowing cytotoxic T cells to lyse infected cells before expression of immune evasive proteins and production of daughter virus (16). gD2 is a major target of neutralizing antibody. Furthermore, heterologous prime-boost immunization has been shown in animal models and clinical trials to elicit broader and more potent immune responses and increased protection (18), including the first positive protection data for a vaccine against HIV-1 (12). Thus, it is likely that antibody and T cell specificities in addition to those that are elicited by mono- or divalent subunit vaccines are needed for optimal neutralization of virus and elimination of infected cells. Indeed, a highly protective Hepatitis A virus vaccine that is formalin-inactivated and aluminum hydroxide adsorbed is one example of an inactivated viral vaccine where this strategy has been successful (24). Additionally, the inactivated HSV-2 boost contains the highly immunogenic Th1 adjuvant MPL and aluminum hydroxide (licensed in Cervarix) which enhances both antibody and T cell responses.

A successful vaccine against HSV disease and shedding should elicit both protective T cell and antibody responses. Combined with recent data demonstrating the presence of T cells specific for the IE proteins ICP0 and ICP4 in asymptomatic/seropositive or in seronegative, HSV-2 exposed populations make these, and other immediate early proteins, highly attractive candidate antigens. Herein, it was discovered that cell immunity against ICP0 and ICP4 and immunity against gD2 can significantly augment the already high-level protection that is elicited by inactivated HSV-2 particles in MPL/Alum adjuvant.

Immunization by DNA prime-inactivated virus particle in adjuvant boost is highly protective against primary and recurrent genital HSV-2. Because of the importance for T cell mediated immunity in the control of HSV-2 infection as shown by both animal models and clinical data, the conserved, essential genes of HSV-2 might also provide T cell-mediated protection for both HSV and HCMV (20). To measure protection against both primary and recurrent infection and disease, a guinea pig model was utilized to evaluate the ability of DNA priming with candidate T cell targets including UL5 (helicase-primase) and UL30 (DNA polymerase) or UL29 (single stranded DNA binding protein) and UL52 (another subunit of the helicase-primase) to augment the protection provided by subsequent boosts with inactivated HSV-2 particles (FI-HSV2). The FI-HSV2 was formulated with a MPL/Alum adjuvant and a plasmid expressing the truncated, secreted form of gD2 (gD2t) was included in the DNA prime. Immunization with gD2t, UL5, UL30 DNAs—FI-HSV2 immunized animals was highly protective against both acute and recurrent lesions with a 97% reduction in the number of days with recurrent lesions after i.vag. challenge when compared with mock controls (FI-Mock). Notably, this group also had the lowest number of days with any level of recurrent disease compared to either the Mock or gD2 subunit control groups. Based on these results with DNA priming with the non-structural virus antigens, T cell responses to relevant T cell antigens of HSV-2 were examined for the ability to augment the protection afforded by the inactivated virus particle vaccine.

The FI-HSV2/MPL/Alum component alone was reasonably protective. However, to simplify the vaccination regimen, the purity and delivery of the FI-HSV2 was enhanced and optimize. Herein, a rapid method for producing HSV-2 was developed that greatly increased the HSV-2 particle to protein ratio. Immunization with these formalin treated particles in MPL/Alhydrogel provided significant 3 Log reductions in vaginal virus titers at the peak of shedding after both short-term (3 week) or long-term (9 week) challenge (21). To test the protection of this preparation against challenge and re-challenge, groups of mice were immunized with the highly pure FI-HSV2 with MPL and alum adjuvants Alhydrogel (Alh) and Adju-Phos (AdP). Significantly, approximately 3 Log reductions in the peak vaginal virus titers relative to mock controls were found in all of the FI-HSV2 groups. Indeed, 5 of 8 of the FI-HSV2/MPL/Alh i.m. group were completely protected against detectable vaginal virus after both challenge and long-term re-challenge. These results are striking in that protective immunity was durable enough to completely protect against virus shedding after a second, long-term lethal challenge.

Formulation with MPL/Alum dramatically enhances immunogenicity and protection from challenge of the inactivated HSV-2 particle vaccine. The inactivated or solubilized envelope glycoprotein HSV-2 vaccines that were previously tested in human subjects were formulated either without adjuvant or with alum only, and these formulations showed only limited immunogenicity and protective efficacy (29). We therefore examined the contribution to immunogenicity and high level protection that the MPL/Alum provides and characterized the types and levels of immune responses in mice. Mice were immunized either i.m. or s.c. with inactivated HSV-2 particles in either phosphate buffered saline (DPBS) or formulated with MPL and Alhydrogel (AlH). We found that formulation of inactivated HSV-2 particles with MPL/Alhydrogel and i.m. delivery resulted in high levels of virus specific CD4+/IFN-g+ T cells, high levels of total IgG; a high-level, balanced IgG1 and IgG2a response; and high levels of virus neutralizing antibodies. This formulation and route resulted in the high levels of protection against acute disease, peak virus shedding, and latent virus load and HSV-2 DNA in DRG, with the majority of mice completely protected against vaginal virus shedding and all mice protected from measurable HSV-2 DNA in ganglia.

An efficacious herpes simplex virus vaccine was developed consisting of highly purified inactivated HSV-2 virions, which contain over 50 different proteins that can generate an immunological response (rather than using just a single glycoprotein). Importantly, the virions are specifically formulated in MPL/Alum adjuvant for the vaccine. The formulation with the adjuvants is the critical component for the highest level of protective efficacy. In some cases, this vaccine can be administered as part of a heterologous prime boost with DNA encoding viral proteins in the priming step.

Protection against HSV-2 shedding in guinea pigs can be significantly augmented by the addition of a recombinant-Herpes virus proteins, such as recombinant gD2 protein to the inactivated HSV-2 particle in the vaccine composition including MPL/Alum. It was also discovered that a higher purity inactivated HSV-2 particle in vaccine composition provides complete protection against lesion development after both challenge and long-term (>4 months) re-challenge and can result in very low-level or undetectable (<4 PFU) virus shedding following both challenges. In addition, complete protection against lesion development, >3 Log reductions in virus replication, and rapid clearance of infectious virus was demonstrated following heterologous challenge with each of 2 additional U.S. strains of HSV-2. The addition to the above vaccine of a 10 to 50-fold excess (the excess is relative to the amount of viral protein in the inactivated virus preparations) of soluble specific proteins encoded by the virus that generate neutralizing antibodies and directed T cell responses, such as envelope and immediate early proteins would increase the protective efficacy.

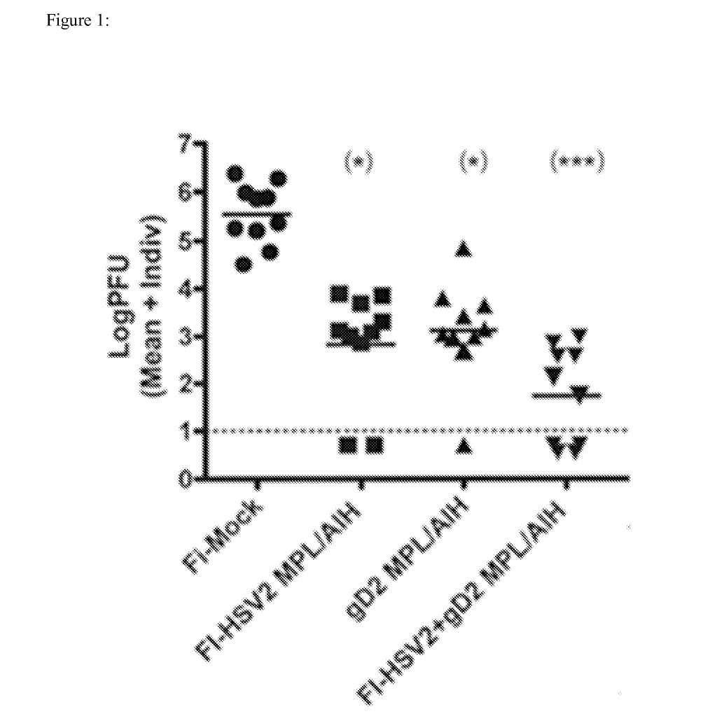

Guinea pigs were vaccinated 3 times as follows: mock virion in MPL/Alum; inactivated virus (FI-HSV2) in MPL/Alum; gD2 in MPL/Alum; and FI-HSV2 plus gD2 in MPL/Alum. They were then challenged intravaginally with HSV-2, and viral shedding was measured. The data show the amount of virus detected at the peak of the infection. See FIG. 1. We found that adding HSV-2 glycoprotein D to the vaccine greatly increases protective efficacy and generates sterilizing immunity in 40% of guinea pigs. The remaining animals show even greater reduction in titer than achieved with the inactivated virus or gD2 alone in adjuvant. Without being bound by any particular theory, the vaccine efficacy appears based on the demonstrated importance of T cells for eradication of infected cells. This likely involves the addition of the HSV-2 proteins (e.g. ICP0 and ICP4) that are key T cell targets found in individuals who are infected but asymptomatic or in HSV-2 exposed/seronegative individuals. It is also likely that a similar response would be obtained in HCMV individuals using homologous immediate early proteins such as IE1 and IE2. The proteins are administered as part of a prime-boost vaccination to augment the protective T cell and antibody responses that are elicited by the inactivated virus alone (plus gD2) in adjuvant. With neutralizing and cellular responses to these antigens, it may be possible to develop a prophylactic and therapeutic vaccine for humans that controls virus both at the mucosal site of entry during the initial infection as well as at the ganglia/mucosal interface during reactivation in already infected individuals.

Using homologous and heterologous prime-boost combinations may optimize T cell responses against ICP0 and ICP4 in the mouse and determine whether protection afforded by the been reported to reactivate latent HSV (13), null mutations were introduced to allow for their eventual clinical use. Specifically, the following mutations were introduced to inhibit function: 1) ICP4, express only the C terminal half that has peptides recognized by seropositive individuals (aa 600 to 1318) and change aa 1063 to 1070, i.e., ADWPADGP to CAWPAAAP which completely inactivates function (6); and 2) ICP0, mutate the RING finger (which confers E3 ubiquitin ligase activity) at C126G and C166A to abrogate latent virus reactivation or transcription (10). The production of high purity HSV-2 virions for inactivation by release from infected Vero cells by dextran sulfate, subsequent purification, and formalin inactivation is as previously described (21, 22).

Formulations for homologous or heterologous prime-boost immunizations are as follows. For DNA vaccination, 20 μg of each endotoxin-free gD2, ICP4, and ICP0 plasmid (or 60 μg of empty pVAX1.2 vector as control) are mixed in Tris-buffered saline for i.m. injection into the tibialis anterior. To formulate inactivated virus, gD2 protein, ICP0 protein, and ICP4 protein with MPL/Alhydrogel, the inactivated HSV-2 (10⁷ PFU equivalents per dose, ca. 0.5-1 μg protein), an equal volume of inactivated Mock, or gD2, ICP0 and ICP4 proteins (5 μg of each protein per dose) is diluted with DPBS and MPL (Sigma, 12.5 μg per dose) and Alhydrogel (125 μg equivalent to ca. 54 μg Al per dose) is added and mixed to facilitate adsorption prior to i.m. immunization as above. Groups of mice are given homologous or heterologous prime-boost immunizations, 4 weeks apart as follows: 1) pVAX—inactivated Mock negative control, homologous groups 2) inactivated HSV-2, 3) ICP0+ICP4 DNAs, 4) gD2+ICP0+ICP4 DNAs, 5) inactivated HSV-2+ICP0+ICP4 protein, 6) inactivated HSV-2+gD2+ICP0+ICP4 protein, or heterologous groups 7) ICP0+ICP4 DNA/inactivated HSV-2, 8) gD2+ICP0+ICP4 DNAs/inactivated HSV-2, 9) ICP0+ICP4 DNA/inactivated HSV-2+ICP0+ICP4 protein, or 10) gD2+ICP0+ICP4 DNAs/inactivated HSV-2+gD2+ICP0+ICP4 protein.

Blood samples (0.2 mL) are collected by orbital bleed: 1) one day prior to the second immunization; 2) one day prior to HSV-2 challenge and 3) one day prior to euthanasia. Immune sera are analyzed for HSV-2 virion-specific IgG, IgG1, and IgG2a ELISA, gD2-specific IgG ELISAs, and neutralizing antibody assays, which are performed essentially as described (22). Vaginal washes and measurement of mucosal IgG and IgA are performed essentially as described (27).

To evaluate cellular immune responses, 4 mice were sacrificed 10 days following the last immunization and another 4 mice were sacrificed 5 days post-infection to assess boosting of primed cellular responses by challenge infection. ELISPOT assay detecting IFN-γ were performed essentially as described (1). Antigen selection for the ELISPOT assay includes 15-mer peptides overlapping by 11 aa of gD2, ICP0, and ICP4 proteins as presented by the vaccines. For groups immunized with inactivated HSV-2, splenocytes are stimulated with UV-inactivated HSV-2 as described (5). To confirm whether T cell responses are either CD4 and/or CD8 in origin, splenocyte populations are enriched for the specific T cell population using Dynal beads (Invitrogen) according to manufacturer's instructions.

On days −7 and −1 relative to challenge, the remaining 8 mice from each group are treated with medroxyprogesterone. Three weeks after the last immunization, mice are intravaginally (i.vag.) challenged with virus (9). Briefly, the mice are inoculated by micropipette-based instillation of 15 μL of virus suspension containing 10 LD₅₀ doses of HSV-2 strain G into the vaginal vault.

Vaginal washes are collected 1 day postchallenge for cytometric bead array analysis of Th1/Th2 cytokines (eBiosciences) to quantify and characterize the rapid mucosal cytokine response to HSV-2 among vaccine groups.

Acute phase disease and survival are assessed as described (21). Animals are scored daily for disease up to 21 days post challenge based on the published severity scale and vaginal swabs are collected on days 2 and 4 postchallenge and stored for high sensitivity (2 PFU per swab) plaque assay on Vero cells (22).

The significance of the survival data is calculated using Kaplan Meier Log Rank test and/or Fisher's exact test. Total disease burden is calculated by disease score area under the curve (AUC). CD4+ and CD8+IFN-g ELISPOT levels, total disease burden, vaginal virus titers, vaginal cytokine levels, and antibody measurements are compared by Kruskal Wallis nonparametric analysis of variance plus Dunn's pairwise nonparametric comparison tests.

The guinea pig model mimics human HSV-2 infection and disease as infected hosts have episodes of recurrent disease and lesions. The frequency of recurrent episodes was shown to increase with the level of HSV-2 DNA load established in DRG (17). However, vaccine induced responses were shown to differentially protect against recurrent disease and virus shedding irrespective of DNA load (4). We found that immunization of guinea pigs with inactivated HSV-2/MPL/Alhydrogel resulted in protective responses against acute disease and reduced latent HSV-2 DNA in DRG (22). Animals that had received prior priming with plasmid DNAs expressing gD2 and conserved, essential HSV-2 proteins UL5 and UL30 showed the greatest reductions in all recurrent HSV-2 disease (22). The advantages of co-immunization with gD2 and ICP0 and ICP4 may be fully realized during the recurrent phase since specific T cell immunity may provide additional control of ganglionic virus or limit the spread of virus during recurrent infection.

Groups of 10 female Hartley guinea pigs/group, 4-6 weeks of age, (300-400 g) are immunized in heterologous or homologous formats. For DNA vaccination, 20 μg of each endotoxin-free gD2, ICP4, and ICP0 plasmid (or 60 μg of empty pVAX1.2 vector as control) are mixed in Tris-buffered saline for i.m. injection into the tibialis anterior. Protein-based vaccines are given by i.m. injection in the tibialis anterior with either 10⁸ PFU equivalents of inactivated HSV-2, an equal volume of inactivated mock, or 5 μg each of gD2, ICP0, and ICP4 proteins. MPL/Alhydrogel groups receive 50 μg MPL and 500 μg of Alhydrogel (equivalent to 238 μg Al). Groups are given homologous or heterologous prime-boost immunizations, 4 weeks apart as follows: 1) pVAX—inactivated Mock negative control, homologous groups 2) inactivated HSV-2, 3) ICP0+ICP4 DNAs, 4) gD2+ICP0+ICP4 DNAs, 5) inactivated HSV-2+ICP0+ICP4 protein, 6) inactivated HSV-2+gD2+ICP0+ICP4 protein, or heterologous groups 7) ICP0+ICP4 DNA/inactivated HSV-2, 8) gD2+ICP0+ICP4 DNAs/inactivated HSV-2, 9) ICP0+ICP4 DNA/inactivated HSV-2+ICP0+ICP4 protein, or 10) gD2+ICP0+ICP4 DNAs/inactivated HSV-2+gD2+ICP0+ICP4 protein.

Blood samples (0.2-0.3 mL) (saphenous vein puncture) are collected: 1) one day prior to each of the two immunizations; 2) one day prior to HSV-2 challenge and 3) one day prior to euthanasia. Immune sera are stored at −20° C. until testing for HSV-2 virion-specific and gD2-specific IgG, IgG1, and IgG2 ELISA titers, and for HSV-2 neutralizing antibody by plaque reduction as described (22). Vaginal washes and measurement of mucosal IgG and IgA are performed essentially as described (27). Three weeks after last immunization, guinea pigs are anesthetized and i.vag. challenged with 5×10⁵ PFU of HSV-2 strain G by means of micropipette and sterile, wide-bore tip.

A vaginal wash is performed on anesthetized guinea pigs on day 1 postchallenge for ELISA determination of IFN-γ and IL-2 levels using a guinea pig specific ELISAs (Uscn Life Sciences, Inc.) following manufacturer's protocols.

Efficacy is evaluated in acute phase (0-14 days post-challenge) lesion formation and viral shedding as described (22). Animals are scored daily for acute lesion formation for 14 days post-challenge based on the published severity scale. To measure acute vaginal viral shedding, vaginal swabs are collected on days 1, 3, 5, 7, 9, and 12 post-challenge and stored in buffer at −80° C. until titration by plaque assay on Vero cell monolayers. To measure recurrent shedding, daily swabs are collected for 21 days starting 4 weeks after challenge and stored for qPCR quantification of HSV-2 DNA. Animals are scored daily for lesion formation during the recurrent phase (days 15-60). At the end of the experiment, the lumbosacral dorsal root ganglia (DRG) are isolated and the amount of HSV-2 latent DNA determined by qPCR as described (22).

Guinea pigs are i.vag. infected with 5×10⁵ PFU of HSV-2 strain G and scored for disease. Surviving animals are randomized into 10 groups by matched disease severity (area under the curve) and immunized on days 21 and 35 as above. Lesions are scored to day 90 postinfection to measure the cumulative number of recurrent lesions and total disease burden in each immunization group. Swabs are collected daily for 1 week before and 3 weeks after the second immunization to measure shedding frequency by qPCR quantification of HSV-2 DNA. At the conclusion of the experiment, animals are sacrificed and DRGs are harvested and stored for qPCR as above.

Protection between groups is assessed by statistical analyses (2-tailed) of the following outcomes of infection. Infection rates, rates of primary lesion development, and rates of recurrent lesion development are each compared by Fisher's exact test. Primary disease burden (as measured by area under the curve (AUC) of disease severity scores, number of recurrent lesion episodes, recurrent disease days, summed recurrent disease scores, vaginal virus shedding levels through day 12, and HSV-2 DNA copies in swabs and DRG are analyzed by Kruskal-Wallis nonparametric one-way analysis of variance followed by pair-wise comparisons using Dunn's multiple comparison tests.

### REFERENCES FOR EXAMPLES

1. Alexander, J., P. Bilsel, M. F. del Guercio, A. Marinkovic-Petrovic, S. Southwood, S. Stewart, G. Ishioka, M. F. Kotturi, J. Botten, J. Sidney, M. Newman, and A. Sette. 2010. Identification of broad binding class I HLA supertype epitopes to provide universal coverage of influenza A virus. Hum Immunol 71:468-74.
2. Ashley, R. L., J. Dalessio, S. Burchett, Z. Brown, S. Berry, K. Mohan, and L. Corey. 1992. Herpes simplex virus-2 (HSV-2) type-specific antibody correlates of protection in infants exposed to HSV-2 at birth. J Clin Invest 90:511-4.
3. Belshe, R. B., P. A. Leone, D. I. Bernstein, A. Wald, M. J. Levin, J. T. Stapleton, I. Gorfinkel, R. L. Morrow, M. G. Ewell, A. Stokes-Riner, G. Dubin, T. C. Heineman, J. M. Schulte, and C. D. Deal. 2012. Efficacy results of a trial of a herpes simplex vaccine. N Engl J Med 366:34-43.
4. Bernstein, D. I., N. Farley, F. J. Bravo, J. Earwood, M. McNeal, J. Fairman, and R. Cardin. 2010. The adjuvant CLDC increases protection of a herpes simplex type 2 glycoprotein D vaccine in guinea pigs. Vaccine 28:3748-53.
5. Braun, R. P., L. Dong, S. Jerome, R. Herber, L. K. Roberts, and L. G. Payne. 2008. Multi-antigenic DNA immunization using herpes simplex virus type 2 genomic fragments. Hum Vaccin 4:36-43.
6. Compel, P., and N. A. DeLuca. 2003. Temperature-dependent conformational changes in herpes simplex virus ICP4 that affect transcription activation. J Virol 77:3257-68.
7. Corey, L. 2007. Herpes simplex virus type 2 and HIV-1: the dialogue between the 2 organisms continues. J. Infect. Dis. 195:1242-1244.
8. Corey, L. 2007. Synergistic copathogens—HIV-1 and HSV-2. N. Engl. J. Med. 356:854-856.
9. Dudek, T., L. C. Mathews, and D. M. Knipe. 2008. Disruption of the U(L)41 gene in the herpes simplex virus 2 dl5-29 mutant increases its immunogenicity and protective capacity in a murine model of genital herpes. Virol. 372:165-175.
10. Ferenczy, M. W., D. J. Ranayhossaini, and N. A. Deluca. 2011. Activities of ICP0 involved in the reversal of silencing of quiescent herpes simplex virus 1. J Virol 85:4993-5002.
11. Freeman, E. E., H. A. Weiss, J. R. Glynn, P. L. Cross, J. A. Whitworth, and R. J. Hayes. 2006. Herpes simplex virus 2 infection increases HIV acquisition in men and women: systematic review and meta-analysis of longitudinal studies. AIDS 20:73-83.
12. Girard, M. P., S. Osmanov, O. M. Assossou, and M. P. Kieny. 2011. Human immunodeficiency virus (HIV) immunopathogenesis and vaccine development: a review. Vaccine 29:6191-218.
13. Halford, W. P., C. D. Kemp, J. A. Isler, D. J. Davido, and P. A. Schaffer. 2001. ICP0, ICP4, or VP16 expressed from adenovirus vectors induces reactivation of latent herpes simplex virus type 1 in primary cultures of latently infected trigeminal ganglion cells. J Virol 75:6143-53.
14. Khanna, K. M., R. H. Bonneau, P. R. Kinchington, and R. L. Hendricks. 2003. Herpes simplex virus-specific memory CD8+ T cells are selectively activated and retained in latently infected sensory ganglia. Immunity 18:593-603.
15. Koelle, D. M., H. Abbo, A. Peck, K. Ziegweid, and L. Corey. 1994. Direct recovery of herpes simplex virus (HSV)-specific T lymphocyte clones from recurrent genital HSV-2 lesions. J Infect Dis 169:956-61.
16. Laing, K. J., L. Dong, J. Sidney, A. Sette, and D. M. Koelle. 2012. Immunology in the Clinic Review Series; focus on host responses: T cell responses to herpes simplex viruses. Clin Exp Immunol 167:47-58.
17. Lekstrom-Himes, J. A., L. Pesnicak, and S. E. Straus. 1998. The quantity of latent viral DNA correlates with the relative rates at which herpes simplex virus types 1 and 2 cause recurrent genital herpes outbreaks. J Virol 72:2760-4.
18. Lu, S. 2009. Heterologous prime-boost vaccination. Curr Opin Immunol 21:346-51.
19. Milligan, G. N., D. I. Bernstein, and N. Bourne. 1998. T lymphocytes are required for protection of the vaginal mucosae and sensory ganglia of immune mice against reinfection with herpes simplex virus type 2. J Immunol 160:6093-100.
20. Morello, C. S., L. A. Kelley, M. W. Munks, A. B. Hill, and D. H. Spector. 2007. DNA immunization using highly conserved murine cytomegalovirus genes encoding homologs of human cytomegalovirus UL54 (DNA polymerase) and UL105 (helicase) elicits strong CD8 T-cell responses and is protective against systemic challenge. J. Virol. 81:7766-7775.
21. Morello, C. S., K. A. Kraynyak, M. S. Levinson, Z. Chen, K. F. Lee, and D. H. Spector. 2012. Inactivated HSV-2 in MPL/alum adjuvant provides nearly complete protection against genital infection and shedding following long term challenge and rechallenge. Vaccine 30:6541-50.
22. Morello, C. S., M. S. Levinson, K. A. Kraynyak, and D. H. Spector. 2011. Immunization with Herpes Simplex Virus Type 2 (HSV-2) Genes Plus Inactivated HSV-2 Is Highly Protective Against Acute and Recurrent HSV-2 Disease. J. Virol. epub January 26.
23. Peng, T., J. Zhu, K. Phasouk, D. M. Koelle, A. Wald, and L. Corey. 2012. An effector phenotype of CD8+ T cells at the junction epithelium during clinical quiescence of herpes simplex virus 2 infection. J Virol 86:10587-96.
24. Rinderknecht, S., M. G. Michaels, M. Blatter, M. Gaglani, W. Andrews, N. Abughali, V. Chandreshekaran, and A. F. Trofa. 2011. Immunogenicity and safety of an inactivated hepatitis A vaccine when coadministered with measles-mumps-rubella and varicella vaccines in children less than 2 years of age. Pediatr Infect Dis J 30:e179-85.
25. Skoberne, M., R. Cardin, A. Lee, A. Kazimirova, V. Zielinski, D. Garvie, A. Lundberg, S. Larson, F. J. Bravo, D. I. Bernstein, J. B. Flechtner, and D. Long. 2013. An adjuvanted herpes simplex virus 2 subunit vaccine elicits a T cell response in mice and is an effective therapeutic vaccine in Guinea pigs. J Virol 87:3930-42.
26. Stanberry, L. R., S. L. Spruance, A. L. Cunningham, D. I. Bernstein, A. Mindel, S. Sacks, S. Tyring, F. Y. Aoki, M. Slaoui, M. Denis, P. Vandepapeliere, G. Dubin, and G. H. V. E. S. Group. 2002. Glycoprotein-D-adjuvant vaccine to prevent genital herpes. N. Engl. J. Med. 347:1652-1661.
27. Tirabassi, R. S., C. I. Ace, T. Levchenko, V. P. Torchilin, L. K. Selin, S. Nie, D. L. Guberski, and K. Yang. 2011. A mucosal vaccination approach for herpes simplex virus type 2. Vaccine 29:1090-8.
28. Wald, A. 2006. Genital HSV-1 infections. Sex Transm Infect 82:189-90.
29. Whitley, R. J. 2001. Herpes Simplex Viruses, p. 2461-2509. In D. M. Knipe and P. M. Howley (ed.), Fields Virology, Fourth ed, vol. 2. Lippincott Williams & Wilkins, Philadelphia.
30. Zhu, J., D. M. Koelle, J. Cao, J. Vazquez, M. L. Huang, F. Hladik, A. Wald, and L. Corey. 2007. Virus-specific CD8+ T cells accumulate near sensory nerve endings in genital skin during subclinical HSV-2 reactivation. J. Exp. Med. 204:595-603.

1. A vaccine composition comprising an inactivated Herpes virus, a recombinant Herpes virus-protein, and a vaccine adjuvant.

2. The vaccine composition of claim 1, wherein said vaccine adjuvant is a lipopolysaccharide (LPS)-adjuvant.

3. The vaccine composition of claim 2, further comprising an aluminum-based mineral salt adjuvant.

4. (canceled)

5. The vaccine composition of claim 1, wherein said vaccine adjuvant is an aluminum-based mineral salt adjuvant.

6. (canceled)

7. (canceled)

8. (canceled)

9. The vaccine composition of claim 1, wherein said recombinant Herpes virus protein is a deactivated-recombinant Herpes virus protein.

10. The vaccine composition of claim 1, wherein said recombinant Herpes virus protein is an envelope protein.

11. The vaccine composition of claim 10, wherein said envelope protein is a glycoprotein.

12. (canceled)

13. (canceled)

14. The vaccine composition of claim 11, wherein said glycoprotein is a glycoprotein D2.

15. (canceled)

16. (canceled)

17. The vaccine composition of claim 1, wherein said recombinant Herpes virus protein is an immediate early Herpes virus protein.

18. The vaccine composition of claim 17, wherein said immediate early Herpes virus protein is ICP0.

19. (canceled)

20. (canceled)

21. The vaccine composition of claim 18, wherein said ICP0 is a modified-ICP0.

22. (canceled)

23. The vaccine composition of claim 21, wherein said modified ICP0 comprises a mutation.

24. (canceled)

25. The vaccine composition of claim 23, wherein said mutation is a point mutation of a residue corresponding to Cys125 or Cys165 of SEQ ID NO: 4.

26. (canceled)

27. (canceled)

28. The vaccine composition of claim 17, wherein said immediate early Herpes virus protein is ICP4.

29. (canceled)

30. (canceled)

31. The vaccine composition of claim 28, wherein said ICP4 is a modified-ICP4.

32. (canceled)

33. (canceled)

34. The vaccine composition of claim 31, wherein said modified-ICP4 comprises a mutation of a residue within a sequence corresponding to residue positions 1063-1070 of SEQ ID NO: 5.

35. (canceled)

36. (canceled)

37. The vaccine composition of claim 17, wherein said immediate early Herpes virus protein is IE2.

38. (canceled)

39. (canceled)

40. (canceled)

41. (canceled)

42. The vaccine composition of claim 1, wherein said vaccine composition is formulated as a pharmaceutical composition comprising a pharmaceutically acceptable excipient.

43. A method of treating a Herpes disease in a subject in need thereof, said method comprising administering a therapeutically effective amount of a vaccine composition according to claim 1.

44. (canceled)

45. (canceled)

46. (canceled)

47. (canceled)

48. (canceled)

49. (canceled)

50. (canceled)

51. A method of preventing a Herpes disease in a subject in need thereof, said method comprising administering a prophylactically effective amount of a vaccine composition according to claim 1.

52.-60. (canceled)
